(19) United States
(12) Patent Application Publication
(10) Pub. No.: US 2026/0340787 A1
(43) Pub. Date: Oct. 29, 2026

(54) SYSTEMS AND METHODS FOR MOTION CORRECTION USING CONDITIONAL DIFFUSION MODELS

(71) Applicant: UNITED IMAGING HEALTHCARE NORTH AMERICA, INC., Houston, TX (US)

(72) Inventor: Zhiyang FU, Houston, TX (US)

(73) Assignee: UNITED IMAGING HEALTHCARE NORTH AMERICA, INC., Houston, TX (US)

(21) Appl. No.: 19/404,068

(22) Filed: Dec. 1, 2025

Related U.S. Application Data
(60) Provisional application No. 63/797,065, filed on Apr. 29, 2025.

(51) Int. Cl. G06T 5/73; G06T 5/60; G06T 7/11; G16H 30/40

(52) U.S. Cl. CPC G06T 5/73; G06T 5/60; G06T 7/11; G16H 30/40

(57) ABSTRACT

The present disclosure provides systems and methods for motion correction using conditional diffusion models. The methods may include performing multiple time steps to process a first medical image to generate a second medical image. A current time step among the time steps includes: performing, based on a randomly generated shift vector, a circular shift on a first intermediate image corresponding to the current time step to generate a second intermediate image, wherein the first intermediate image is generated based on the first medical image or a third intermediate image corresponding to a previous time step; dividing the second intermediate image into image patches; generating a third intermediate image corresponding to the current time step based on the image patches, the shift vector, and a trained conditional diffusion model; and designating the third intermediate image as the second medical image or proceeding to a next time step.

### CROSS-REFERENCE TO RELATED APPLICATIONS

This application claims priority to U.S. Provisional Patent Application No. 63/797,065, filed on Apr. 29, 2025, the contents of which are incorporated herein by reference.

### TECHNICAL FIELD

The present disclosure relates to the field of medical imaging, and more particularly, relates to systems and methods for motion correction using conditional diffusion models.

### BACKGROUND

With the advancement of science and technology, deep learning techniques have become increasingly significant in the field of medical imaging, with applications including image denoising, super-resolution, image reconstruction, artifact reduction, etc. Recently, conditional diffusion models have demonstrated the capability to generate high-quality and high-resolution images by learning from the distribution of training datasets. Accordingly, it is desirable to provide systems and methods that apply the conditional diffusion models in the field of medical imaging (e.g., coronary computed tomography angiography (CCTA)), so as to enhance the processing accuracy and efficiency of medical images.

### SUMMARY

According to an aspect of the present disclosure, a method for motion correction is provided. The method may be implemented on a computing device having at least one processor and at least one storage device. The method may include performing multiple time steps to process a first medical image to generate a second medical image. A current time step among the time steps may include performing, based on a randomly generated shift vector, a circular shift on a first intermediate image corresponding to the current time step to generate a second intermediate image. The first intermediate image may be generated based on the first medical image or a third intermediate image corresponding to a previous time step. The current time step among the time steps may include dividing the second intermediate image into image patches. The current time step among the time steps may include generating a third intermediate image corresponding to the current time step based on the image patches, the shift vector, and a trained conditional diffusion model. The current time step among the time steps may include designating the third intermediate image as the second medical image or proceeding to a next time step.

In some embodiments, the generating a third intermediate image corresponding to the current time step based on the image patches, the shift vector, and a trained conditional diffusion model may include: for each of the image patches, generating a processed image patch by inputting the image patch into the trained conditional diffusion model; generating a fourth intermediate image by splicing the processed image patches; and generating the third intermediate image corresponding to the current time step by performing, based on the shift vector, a reverse circular shift on the fourth intermediate image.

In some embodiments, the generating a processed image patch by inputting the image patch into the trained conditional diffusion model may include: generating the processed image patch by inputting the image patch and at least one of positional information or size information of the image patch into the trained conditional diffusion model.

In some embodiments, the first medical image may be a two-dimensional (2D) image corresponding to an anatomical plane, and the generating a processed image patch by inputting the image patch into a trained conditional diffusion model may include: determining one or more adjacent image patches corresponding to the image patch from one or more adjacent medical images of the first medical image, wherein the one or more adjacent medical images correspond to one or more neighboring anatomical planes of the anatomical plane; and generating the processed image patch by inputting the image patch and the one or more adjacent image patches into the trained conditional diffusion model.

In some embodiments, the generating a processed image patch by inputting the image patch into the trained conditional diffusion model may include: generating 180° parallel-beam sinogram data by performing forward projection on the image patch; dividing the 180° parallel-beam sinogram data into multiple sinogram datasets corresponding to different partial angular ranges; generating partial angle reconstruction (PAR) image blocks by performing back-projection on the sinogram datasets; and generating the processed image patch by inputting the PAR image blocks into the trained conditional diffusion model.

In some embodiments, the generating a processed image patch by inputting the image patch into the trained conditional diffusion model may include: obtaining a deformation vector field (DVF) by inputting the image patch into the trained conditional diffusion model; and generating the processed image patch by processing the DVF and the image patch using a spatial transformer layer (STL), wherein the trained conditional diffusion model is trained end-to-end within a supervised learning framework that incorporates an initial conditional diffusion model and the STL.

In some embodiments, the first intermediate image may be generated by concatenating the first medical image or the third intermediate image corresponding to the previous time step with a random noise image.

In some embodiments, the first intermediate image may be generated by concatenating the first medical image or the third intermediate image corresponding to the previous time step further with a positional coordinate array corresponding to the first medical image.

In some embodiments, the trained conditional diffusion model may be generated by training an initial conditional diffusion model using training samples, each of the training samples may include a first sample image patch and a corresponding second sample image patch, which may be generated by: obtaining a first sample image and a second sample image of a sample subject; generating a first shifted sample image by performing, based on a randomly generated sample shift vector, a circular shift on the first sample image; generating a second shifted sample image by performing, based on the sample shift vector, a circular shift on the second sample image; and extracting the first sample image patch from the first shifted sample image and the corresponding second sample image patch from the second shifted sample image.

In some embodiments, the first sample image and the second sample image may be acquired using X-ray imaging, a loss function used to train the initial conditional diffusion model may include a first loss term and a second loss term, the first loss term may relate to a difference between a predicted image patch output by the initial conditional diffusion model and the second sample image patch, and the second loss term relates to a difference between back-projection data of the predicted image patch and back-projection data of the first sample image patch.

In some embodiments, the designating the third intermediate image as the second medical image or proceeding to a next time step may include: in response to determining that a preset count of time steps has been completed, evaluating whether the third intermediate image satisfies a quality condition; in response to determining that the third intermediate image satisfies the quality condition, designating the third intermediate image as the second medical image; or in response to determining that the third intermediate image does not satisfy the quality condition, post-processing the third intermediate image to generate the second medical image or proceeding to the next time step.

According to another aspect of the present disclosure, a method for motion correction is provided. The method may be implemented on a computing device having at least one processor and at least one storage device. The method may include obtaining a blood vessel image. The method may include extracting image patches from the blood vessel image. Each of the image patches may be centered on a center point of blood vessels in the blood vessel image. The method may include, for each of the image patches, generating a corrected image patch by inputting the image patch into a trained conditional diffusion model. The method may further include generating a corrected blood vessel image based on the blood vessel image and the corrected image patches. The corrected blood vessel image may have an improved quality than the blood vessel image.

In some embodiments, the blood vessel image may be a two-dimensional (2D) image corresponding to an anatomical plane, and for each of the image patches, the generating a corrected image patch by inputting the image patch into a trained conditional diffusion model may include: for each of the image patches, determining one or more adjacent image patches corresponding to the image patch from one or more adjacent blood vessel images of the blood vessel image, wherein the one or more adjacent blood vessel images correspond to one or more neighboring anatomical planes of the anatomical plane; and generating the corrected image patch by inputting the image patch and the one or more adjacent image patches into the trained conditional diffusion model.

In some embodiments, the generating a corrected image patch by inputting the image patch into a trained conditional diffusion model may include: generating 180° parallel-beam sinogram data by performing forward projection on the image patch; dividing the 180° parallel-beam sinogram data into multiple sinogram datasets corresponding to different partial angular ranges; generating partial angle reconstruction (PAR) image blocks by performing back-projection on the sinogram datasets; and generating the corrected image patch by inputting the PAR image blocks into the trained conditional diffusion model.

In some embodiments, the generating a corrected image patch by inputting the image patch into a trained conditional diffusion model may include: obtaining a deformation vector field (DVF) by inputting the image patch into the trained conditional diffusion model; and generating the corrected image patch by processing the DVF and the image patch using a spatial transformer layer (STL), wherein the trained conditional diffusion model is trained end-to-end within a supervised learning framework that incorporates an initial conditional diffusion model and the STL.

In some embodiments, the corrected blood vessel image may include less motion artifacts than the blood vessel image, the trained conditional diffusion model may be generated by training an initial conditional diffusion model using training samples, each of the training samples may include a first sample image patch and a second sample image patch, the first sample image patch may be used as a training input and extracted from a simulated artifact-corrupted image of sample blood vessels corresponding to a middle motion phase among multiple motion phases; the second sample image patch may be used as a training label and extracted from a simulated artifact-free image of the sample blood vessels corresponding to the middle motion phase.

In some embodiments, the simulated artifact-free image corresponding to the middle motion phase may be generated by: generating a segmentation result of the sample blood vessels by segmenting a sample blood vessel image of the sample blood vessels, the sample blood vessel image including artifacts; generating an inpainted image without the sample blood vessels by removing the sample blood vessels from the sample blood vessel image; generating a transformed segmentation result corresponding to the middle motion phase by transforming the segmentation result; and generating the simulated artifact-free image corresponding to the middle motion phase by inserting the transformed segmentation result into the inpainted image.

In some embodiments, the sample blood vessels may be coronal arteries, and the segmentation result may be transformed based on a first DVF, a second DVF, and a third DVF, the first DVF may be related to the cardiac motion in the middle motion phase, the second DVF may be related to the respiratory motion in the middle motion phase, and the third DVF may be related to random jitter.

In some embodiments, the simulated artifact-corrupted image may be generated based on simulated artifact-free images corresponding to the motion phases by: for each of the simulated artifact-free images, generating sinogram data by performing a fan-beam simulation scan based on the simulated artifact-free image; stacking the sinogram data corresponding to the simulated artifact-free images; and generating the simulated artifact-corrupted image corresponding to the middle motion phase by reconstructing the stacked sinogram data.

According to still another aspect of the present disclosure, a system for motion correction is provided. The system may include at least one storage device including a set of instructions; and at least one processor in communication with the at least one storage device. When executing the set of instructions, the at least one processor may be directed to cause the system to perform operations. The operations may include performing multiple time steps to process a first medical image to generate a second medical image. A current time step among the time steps may include performing, based on a randomly generated shift vector, a circular shift on a first intermediate image corresponding to the current time step to generate a second intermediate image. The first intermediate image may be generated based on the first medical image or a third intermediate image corresponding to a previous time step. The current time step among the time steps may include dividing the second intermediate image into image patches. The current time step among the time steps may include generating a third intermediate image corresponding to the current time step based on the image patches, the shift vector, and a trained conditional diffusion model. The current time step among the time steps may include designating the third intermediate image as the second medical image or proceeding to a next time step.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further illustrated in terms of exemplary embodiments. These exemplary embodiments are described in detail according to the drawings. These embodiments are non-limiting exemplary embodiments, in which like reference numerals represent similar structures, wherein:

FIG. 1 is a schematic diagram illustrating an exemplary imaging system according to some embodiments of the present disclosure;

FIG. 2 is a block diagram illustrating an exemplary processing device according to some embodiments of the present disclosure;

FIG. 3 is a schematic diagram illustrating an exemplary process for generating a second medical image according to some embodiments of the present disclosure;

FIG. 4A is a schematic diagram illustrating an exemplary first intermediate image according to some embodiments of the present disclosure;

FIG. 4B is a schematic diagram illustrating an exemplary second intermediate image according to some embodiments of the present disclosure;

FIG. 4C is a schematic diagram illustrating an exemplary second intermediate image after patchified according to some embodiments of the present disclosure;

FIG. 4D is a schematic diagram illustrating exemplary image patches according to some embodiments of the present disclosure;

FIG. 5 is a schematic diagram illustrating an exemplary process for generating a third intermediate image corresponding to a current time step according to some embodiments of the present disclosure;

FIG. 6 is a schematic diagram illustrating an exemplary process for generating a processed image patch through a trained conditional diffusion model according to some embodiments of the present disclosure;

FIG. 7 is a schematic diagram illustrating an exemplary architecture of a trained conditional diffusion model according to some embodiments of the present disclosure;

FIG. 8 is a schematic diagram illustrating an exemplary architecture of a trained conditional diffusion model according to some embodiments of the present disclosure;

FIG. 9 is a flowchart illustrating an exemplary process for generating a processed image patch according to some embodiments of the present disclosure;

FIG. 10 is a flowchart illustrating an exemplary process for generating a processed image patch according to some embodiments of the present disclosure;

FIG. 11 is a flowchart illustrating an exemplary process for generating a corrected blood vessel image according to some embodiments of the present disclosure;

FIG. 12 is a schematic diagram illustrating an exemplary process for generating a corrected CCTA image according to some embodiments of the present disclosure;

FIG. 13 is a flowchart illustrating an exemplary process for generating a simulated artifact-free image according to some embodiments of the present disclosure;

FIG. 14 is a schematic diagram illustrating an exemplary process for generating a simulated artifact-free image according to some embodiments of the present disclosure;

FIG. 15 is a flowchart illustrating an exemplary process for generating a simulated artifact-corrupted image according to some embodiments of the present disclosure;

FIG. 16 is a schematic diagram illustrating an exemplary process for generating a simulated artifact-corrupted image according to some embodiments of the present disclosure;

FIG. 17 is a schematic diagram illustrating exemplary motion correction results according to some embodiments of the present disclosure;

FIG. 18 is a schematic diagram illustrating exemplary 3D segmentation results according to some embodiments of the present disclosure; and

FIGS. 19A-19D are schematic diagrams illustrating box plots with scattered data points of Dice score according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system,” “device,” “unit,” and/or “module” as used herein is a manner used to distinguish different components, elements, parts, sections, or assemblies at different levels. However, if other words serve the same purpose, the words may be replaced by other expressions.

As shown in the present disclosure and claims, the words “one,” “a,” “a kind,” and/or “the” are not especially singular but may include the plural unless the context expressly suggests otherwise. In general, the terms “comprise,” “comprises,” “comprising,” “include,” “includes,” and/or “including,” merely prompt to include operations and elements that have been clearly identified, and these operations and elements do not constitute an exclusive listing. The methods or devices may also include other operations or elements.

It will be understood that, although the terms “first,” “second,” “third,” “fourth,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

Spatial and functional relationships between elements are described using various terms, including “connected,” “engaged,” “interfaced,” and “coupled.” Unless explicitly described as being “direct,” when a relationship between first and second elements is described in the present disclosure, that relationship includes a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.). As used herein, the term “and/or” includes any and all combinations of one or more of the associated listed items.

The flowcharts used in the present disclosure illustrate operations that systems implement according to some embodiments of the present disclosure. It should be understood that the previous or subsequent operations may not be accurately implemented in order. Instead, each step may be processed in reverse order or simultaneously. Meanwhile, other operations may also be added to these processes, or a certain step or several steps may be removed from these processes.

In the present disclosure, the subject may include a biological object and/or a non-biological object. The biological object may be a human being, an animal, a plant, or a specific portion, organ, and/or tissue thereof. For example, the subject may include the head, the neck, the thorax, the heart, the stomach, a blood vessel, a soft tissue, a tumor, a nodule, or the like, or any combination thereof. In some embodiments, the subject may be a man-made composition of organic and/or inorganic matters that are with or without life. The terms “object” and “subject” are used interchangeably in the present disclosure.

In the present disclosure, the term “image” may refer to a two-dimensional (2D) image, a three-dimensional (3D) image, or a four-dimensional (4D) image (e.g., a time series of 3D images). In some embodiments, the term “image” may refer to an image of a region (e.g., a region of interest (ROI)) of a subject. In some embodiments, the image may be a medical image, an optical image, etc.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an ROI from the image may be referred to as a segmentation of the ROI for brevity.

For illustration purposes, the following description is provided to help better understanding an imaging process. It is understood that this is not intended to limit the scope of the present disclosure. For persons having ordinary skills in the art, a certain amount of variations, changes and/or modifications may be deducted under guidance of the present disclosure. Those variations, changes and/or modifications do not depart from the scope of the present disclosure.

The present disclosure relates to systems and methods for motion correction using conditional diffusion models. The methods may include performing multiple time steps to process a first medical image to generate a second medical image. A current time step among the time steps may include performing, based on a randomly generated shift vector, a circular shift on a first intermediate image corresponding to the current time step to generate a second intermediate image. The first intermediate image may be generated based on the first medical image or a third intermediate image corresponding to a previous time step. The current time step among the time steps may also include dividing the second intermediate image into image patches. The current time step among the time steps may include generating a third intermediate image corresponding to the current time step based on the image patches, the shift vector, and a trained conditional diffusion model. The current time step among the time steps may further include designating the third intermediate image as the second medical image or proceeding to a next time step.

By inputting the image patches instead of the entire image into the conditional diffusion model, the computational resources required for the application of the conditional diffusion model can be significantly reduced, thereby enhancing the computational efficiency. Furthermore, a cyclic shift mechanism is introduced to the training and reference of the conditional diffusion model, thereby reducing or eliminating the blocking artifacts that are caused by the image division.

Furthermore, the systems and methods can be applied to the correction of blood vessel images, thereby removing motion artifacts from the blood vessel images and improving the quality of the blood vessel images.

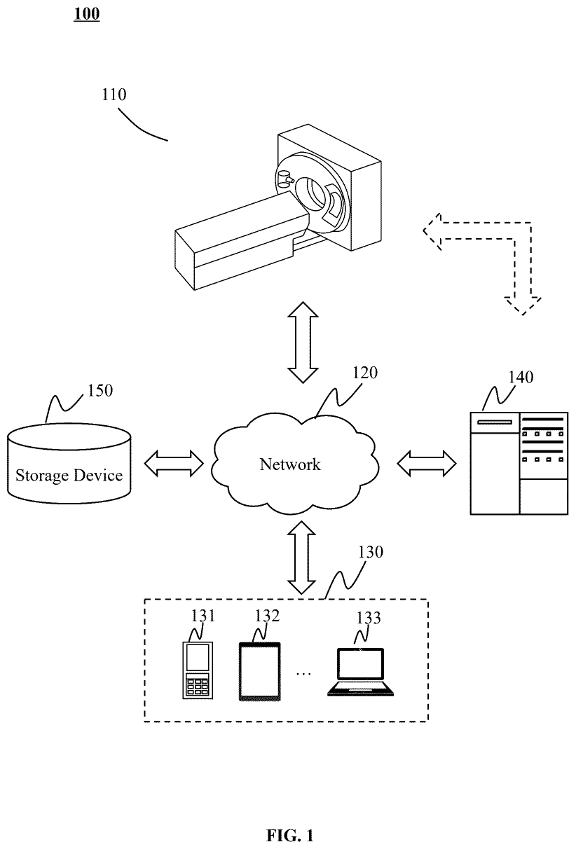

FIG. 1 is a schematic diagram illustrating an exemplary imaging system 100 according to some embodiments of the present disclosure.

As shown in FIG. 1, in some embodiments, the imaging system 100 may include an imaging device 110, a network 120, one or more terminal devices 130, a processing device 140, and a storage device 150. In some embodiments, the components of the imaging system 100 may be connected to each other via the network 120. Alternatively or additionally, the components of the imaging system 100 may be directly connected to each other. Merely by way of example, the imaging device 110 may be connected to the processing device 140 through the network 120, as illustrated in FIG. 1. As another example, the imaging device 110 may be connected to the processing device 140 directly.

The imaging device 110 may be configured to generate or provide image data by scanning a subject or at least a part of the subject. For example, the imaging device 110 may scan the subject or a portion thereof that is located within its detection region and generate the image data (e.g., a medical image) relating to the subject or the portion thereof. In some embodiments, the imaging device 110 may include a single modality imaging device. For example, the imaging device 110 may include a computed tomography (CT) device, an X-ray imaging device, an optical coherence tomography (OCT) imaging system, an ultrasound imaging device, an intravascular ultrasound (IVUS) imaging system, a near-infrared spectroscopy (NIRS) imaging system, a digital subtraction angiography (DSA) system, a positron emission tomography (PET) device, a single-photon emission computed tomography (SPECT) device, a magnetic resonance imaging (MRI) device, a thermal texture maps (TTM) device, a medical electronic endoscope (MEE) device, or the like, or any combination thereof. In some embodiments, the imaging device 110 may include a multi-modality imaging device. Exemplary multi-modality imaging devices may include a positron emission tomography-computed tomography (PET-CT) device, a digital subtraction angiography-magnetic resonance imaging (DSA-MRI) system, a single-photon emission computed tomography-computed tomography (SPECT-CT) device, etc. The multi-modality scanner may perform multi-modality imaging simultaneously or in sequence. For example, the PET-CT device may generate structural X-ray CT image data and functional PET image data simultaneously or in sequence.

The network 120 may include any suitable network that can facilitate the exchange of information and/or data for the imaging system 100. In some embodiments, one or more components (e.g., the imaging device 110, the terminal(s) 130, the processing device 140, the storage device 150, etc.) of the imaging system 100 may communicate information and/or data with one or more other components of the imaging system 100 via the network 120. For example, the processing device 140 may obtain the image data from the imaging device 110 via the network 120. As another example, the processing device 140 may obtain user instructions from the terminal(s) 130 via the network 120. In some embodiments, the network 120 may include one or more network access points. For example, the network 120 may include wired and/or wireless network access points such as base stations and/or internet exchange points through which one or more components of the imaging system 100 may be connected to the network 120 to exchange data and/or information.

The terminal(s) 130 may include a mobile device 131, a tablet computer 132, a laptop computer 133, or the like, or any combination thereof. In some embodiments, the mobile device 131 may include a smart home device, a wearable device, a mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, a footgear, eyeglasses, a helmet, a watch, clothing, a backpack, a smart accessory, or the like, or any combination thereof. In some embodiments, the mobile device may include a mobile phone, a personal digital assistant (PDA), a gaming device, a navigation device, a point of sale (POS) device, a laptop, a tablet computer, a desktop, or the like, or any combination thereof. In some embodiments, the virtual reality device and/or the augmented reality device may include a virtual reality helmet, virtual reality glasses, a virtual reality patch, an augmented reality helmet, augmented reality glasses, an augmented reality patch, or the like, or any combination thereof. In some embodiments, the terminal(s) 130 may be part of the processing device 140.

The processing device 140 may process data and/or information obtained from one or more components (the imaging device 110, the terminal(s) 130, and/or the storage device 150) of the imaging system 100. For example, the processing device 140 may perform multiple time steps to process a first medical image to generate a second medical image. As another example, the processing device 140 may generate a corrected blood vessel image by correcting image patches extracted from a blood vessel image. In some embodiments, the processing device 140 may be a single server or a server group. The server group may be centralized or distributed. In some embodiments, the processing device 140 may be local or remote. For example, the processing device 140 may access information and/or data stored in the imaging device 110, the terminal(s) 130, and/or the storage device 150 via the network 120. As another example, the processing device 140 may be directly connected to the imaging device 110, the terminal(s) 130, and/or the storage device 150 to access stored information and/or data. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the processing device 140 may be implemented by a computing device. For example, the computing device may include a processor, a storage device, an input/output (I/O), and a communication port. The processor may execute computer instructions (e.g., program codes) and perform functions of the processing device 140 in accordance with the techniques described herein. The computer instructions may include, for example, routines, programs, objects, components, data structures, procedures, modules, and functions, which perform particular functions described herein. In some embodiments, the processing device 140, or a portion of the processing device 140 may be implemented by a portion of the terminal(s) 130.

The storage device 150 may store data/information obtained from the imaging device 110, the terminal(s) 130, and/or any other component of the imaging system 100. In some embodiments, the storage device 150 may include a mass storage, a removable storage, a volatile read-and-write memory, a read-only memory (ROM), or the like, or any combination thereof. For example, the mass storage may include a magnetic disk, an optical disk, a solid-state drive, etc. The removable storage may include a flash drive, a floppy disk, an optical disk, a memory card, a zip disk, a magnetic tape, etc. In some embodiments, the storage device 150 may store one or more programs and/or instructions to perform exemplary methods described in the present disclosure.

In some embodiments, the storage device 150 may be connected to the network 120 to communicate with one or more other components in the imaging system 100 (e.g., the processing device 140, the terminal(s) 130, etc.). One or more components in the imaging system 100 may access the data or instructions stored in the storage device 150 via the network 120. In some embodiments, the storage device 150 may be directly connected to or communicate with one or more other components in the imaging system 100 (e.g., the processing device 140, the terminal(s) 130, etc.). In some embodiments, the storage device 150 may be part of the processing device 140.

It should be noted that the above description is merely provided for the purposes of illustration, and is not intended to limit the scope of the present disclosure. For persons having ordinary skills in the art, multiple variations and modifications may be made under the teachings of the present disclosure. Features, structures, methods, and other characteristics of the exemplary embodiments described herein may be combined in various ways to obtain additional and/or alternative exemplary embodiments. For example, the imaging system 100 may also include a plurality of other components, such as a patient positioning unit, a data acquisition electronic, a power supply, and other devices or units. However, those variations and modifications do not depart from the scope of the present disclosure.

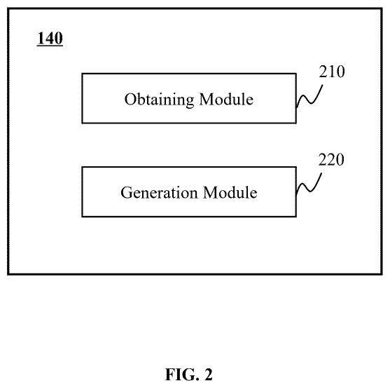

FIG. 2 is a block diagram illustrating an exemplary processing device 140 according to some embodiments of the present disclosure. In some embodiments, the processing device 140 may be in communication with a computer-readable storage medium (e.g., the storage device 150 illustrated in FIG. 1) and execute instructions stored in the computer-readable storage medium. The processing device 140 may include an obtaining module 210 and a generation module 220.

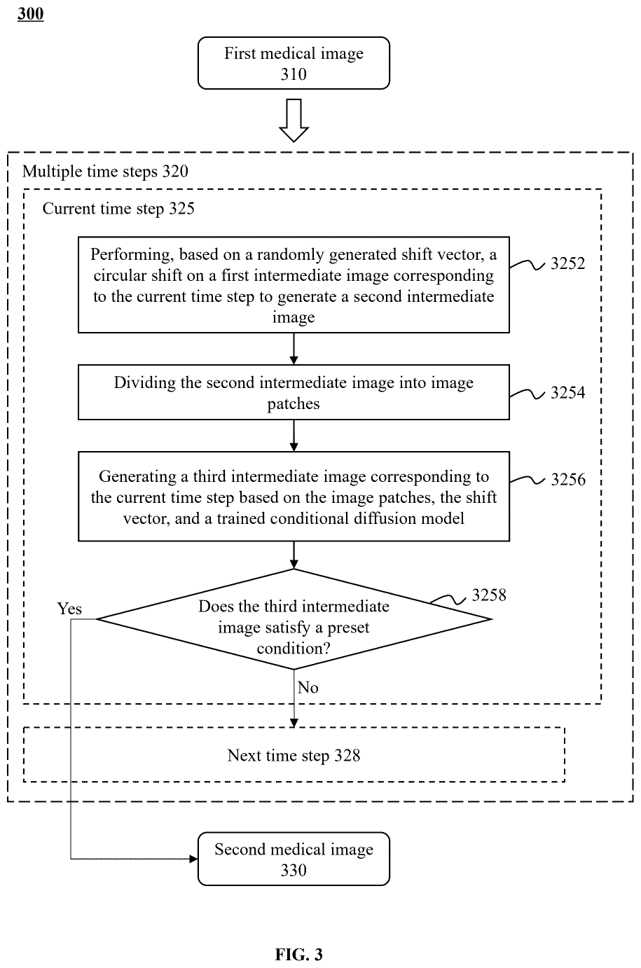

The obtaining module 210 may be configured to obtain a first medical image. The first medical image refers to a medical image of a subject that needs to be processed (e.g., denoised, smoothed, etc.). More descriptions regarding the obtaining of the first medical image may be found elsewhere in the present disclosure. See, e.g., FIG. 3 and relevant descriptions thereof.

The generation module 220 may be configured to generate a second medical image by performing multiple time steps to process the first medical image. The second medical image refers to a medical image after processing. In some embodiments, the second medical image may have a higher image quality than the first medical image. A time step refers to an iteration or a round that uses a trained conditional diffusion model to process the first medical image. The time step may represent a stage in a reverse denoising process and condition the trained conditional diffusion model to perform the appropriate amount of denoising (or noising) at that particular stage. More descriptions regarding the generation of the second medical image may be found elsewhere in the present disclosure. See, e.g., FIG. 3 and relevant descriptions thereof.

It should be noted that the above description regarding the processing device 140 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. For example, the processing device 140 may include a storage module configured to store data generated by the above-mentioned modules of the processing device 140. As another example, one or more modules may be integrated into a single module to perform the functions thereof.

FIG. 3 is a schematic diagram illustrating an exemplary process 300 for generating a second medical image according to some embodiments of the present disclosure.

Conditional diffusion models have emerged and surpassed generative adversarial networks (GANs) in image generation and synthesis tasks. The conditional diffusion models are designed to map, based on conditional information, any data distribution from Gaussian noise through an iterative denoising process. When guided with known priors, the conditional diffusion models have found success in a great many applications, including, but not limited to, text-to-image generation, image denoising, and image restoration. However, unlike convolutional neural networks (CNNs) or GANs, the conditional diffusion models require hundreds or even thousands of evaluations (e.g., multiple time steps or multiple iterations) of the same network to generate a single image. While some works have been proposed to accelerate the sampling process of the conditional diffusion models, it is computationally prohibitive to train the conditional diffusion models for high-resolution data, high-dimensional data, or both, for example, medical images. Furthermore, as the problem size scales, diffusion sampling quickly becomes memory-limited as well.

Therefore, to reduce computational memory cost of the conditional diffusion models and enable their application in medical image processing, the process 300 may be performed.

As illustrated in FIG. 3, multiple time steps 320 may be performed to process a first medical image 310 to generate a second medical image 330.

The first medical image 310 refers to a medical image of a subject that needs to be processed (e.g., denoised, smoothed, etc.). For example, the first medical image 310 may include an image with artifacts, a low-resolution image, an image with a low sampling rate, an image corresponding to a short scanning time, etc. In some embodiments, the first medical image 310 may be a 2D image, a 3D image, or a 4D image. For example, the first medical image 310 may be a 2D image corresponding to an anatomical plane (e.g., a transverse section, a coronal section, or a cross section).

In some embodiments, the processing device 140 (e.g., the obtaining module 210) may obtain the first medical image 310 from an imaging device (e.g., the imaging device 110) or a storage device (e.g., the storage device 150, a database, or an external storage) that stores the first medical image 310. Alternatively, the processing device 140 may obtain the first medical image 310 by reconstructing image data corresponding to the first medical image 310.

The second medical image 330 refers to a medical image after processing. For example, the second medical image 330 may be the processed first medical image after the time steps 320 are performed. As another example, the second medical image 330 may include an image after artifact correction, a high-resolution image, an image with a high sampling rate, an image corresponding to a long scanning time, etc.

Therefore, the second medical image 330 may have a higher image quality than the first medical image 310. As used herein, the image quality of an image may be measured by one or more image quality parameters, such as, the resolution, a noise ratio, a signal-to-noise ratio, an artifact level, etc. For example, the second medical image 330 may include less artifacts (e.g., motion artifacts) than the first medical image 310. For instance, the first medical image 310 may be an artifact-corrupted image of blood vessels and the second medical image 330 may be an artifact-free image of the blood vessels. As another example, the second medical image 330 may correspond to a higher radiation dose than the first medical image 310. As still another example, the second medical image 330 may correspond to a higher sampling rate than the first medical image 310. As yet another example, the second medical image 330 may correspond to a longer scanning time than the first medical image 310.

In some embodiments, the processing device 140 (e.g., the generation module 220) may generate the second medical image 330 by performing the time steps 320 to process the first medical image 310.

A time step refers to an iteration or a round that uses a trained conditional diffusion model to process the first medical image 310. The time step may represent a stage in a reverse denoising process and condition the trained conditional diffusion model to perform the appropriate amount of denoising at that particular stage.

For illustration purposes, a current time step 325 among the time steps 320 is taken as an example and described below. The current time step 325 may include the following operations.

In 3252, the processing device 140 (e.g., the generation module 220) may perform, based on a randomly generated shift vector, a circular shift on a first intermediate image corresponding to the current time step 325 to generate a second intermediate image.

The first intermediate image refers to an initial image to be processed in the current time step 325.

In some embodiments, the first intermediate image may be generated based on the first medical image 310 or a third intermediate image corresponding to a previous time step (also referred to as a previous third intermediate image). For example, if the current time step 325 is the first time step among the time steps 320, the first intermediate image may be generated based on the first medical image 310. For instance, the processing device 140 may designate the first medical image 310 as the first intermediate image. As another example, the processing device 140 may pre-process the first intermediate image, and designate the pre-processed first medical image as the first intermediate image.

As another example, if the current time step 325 is a time step other than the first time step, the first intermediate image may be generated based on the previous third intermediate image. For instance, the processing device 140 may designate the previous third intermediate image as the first intermediate image. As another example, the processing device 140 may pre-process the previous third intermediate image, and designate the pre-processed previous third intermediate image as the first intermediate image.

In some embodiments, the first intermediate image may be generated further based on a random noise image. The random noise image refers to an image including random and disordered pixel (or voxel) values. For example, the random noise image may follow a certain statistical distribution, such as, the Gaussian distribution (also referred to as normal distribution). Merely by way of example, the processing device 140 may generate the first intermediate image by concatenating the first medical image 310 or the previous third intermediate image with the random noise image. The concatenating operation may be performed along the channel dimension.

In some embodiments, the first intermediate image may be generated further based on a positional coordinate array corresponding to the first medical image 310. The positional coordinate array is an array including a positional coordinate of each pixel (or voxel) in the first medical image 310. For example, the first intermediate image may be generated by concatenating the first medical image 310 or the previous third intermediate image with the positional coordinate array corresponding to the first medical image 310. As another example, the first intermediate image may be generated by concatenating the first medical image 310 or the previous third intermediate image with the random noise image and the positional coordinate array corresponding to the first medical image 310.

The circular shift on the first intermediate image refers to a geometric transformation of the first intermediate image where the pixels (or voxels) that are shifted out of the image boundary on one side are reintroduced on the opposite side. For example, each pixel (or voxel) in the first intermediate image may be shifted based on the randomly generated shift vector.

The randomly generated shift vector refers to a shift vector for each pixel (or voxel) in the first intermediate image that is randomly generated. A dimension of the randomly generated shift vector may relate to a dimension of the first intermediate image. For example, if the first intermediate image is a two-dimensional image, the randomly generated shift vector may be a two-dimensional vector. As another example, if the first intermediate image is a three-dimensional image, the randomly generated shift vector may be a three-dimensional vector.

In some embodiments, each dimension of the randomly generated shift vector may correspond to one dimension of the first intermediate image and include a shift direction (e.g., a positive direction, a negative direction) and a displacement (e.g., a count of pixels) along the corresponding dimension of the first intermediate image. For example, the displacement of each dimension of the randomly generated shift vector may be within a range from 0 to a patch size (which will be described below).

Merely by way of example, for a two-dimensional first intermediate image, the randomly generated shift vector may be a two-dimensional vector including a first element and a second element. The first element corresponds to the horizontal dimension of the first intermediate image and the second element corresponds to the vertical dimension of the first intermediate image. If the first element is +3 and the second element is −2, each pixel in the first intermediate image may be shifted three pixels along the positive direction of the horizontal dimension and two pixels along the negative direction of the vertical dimension, so as to generate the second intermediate image.

By circularly shifting the first intermediate image based on the randomly generated shift vector, positions of edges of the subject in the second intermediate image are different in each time step, which can eliminate or reduce blocking artifacts in the resulting second medical image 330, thereby improving the accuracy of the image prediction.

In 3254, the processing device 140 (e.g., the generation module 220) may divide the second intermediate image into image patches.

An image patch may be a small region extracted from the second intermediate image and include multiple consecutive pixels (or voxels) in the second intermediate image.

In some embodiments, patch sizes of the image patches may be the same. That is, the processing device 140 may uniformly divide (e.g., patchify) the second intermediate image into the image patches. For example, if a size of the second intermediate image is 1024*1024 (pixels), the patch size may be 64*64 or 128*128. In some embodiments, the patch size of each image patch (or the count of the image patches) may be determined based on an actual requirement (e.g., a processing time requirement, a processing memory requirement, a processing accuracy requirement, etc.).

In some embodiments, each of the image patches may not be overlapped with other image patches. For example, each pixel (or voxel) in the second intermediate image can appear only once in the image patches.

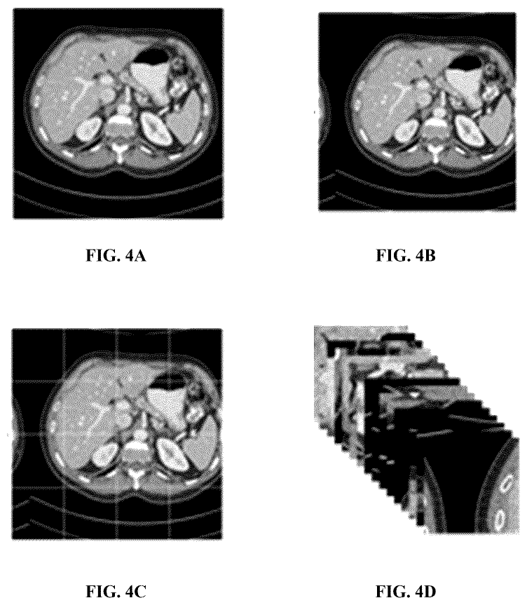

Merely by way of example, referring to FIGS. 4A-4D, FIG. 4A is a schematic diagram illustrating an exemplary first intermediate image according to some embodiments of the present disclosure. FIG. 4B is a schematic diagram illustrating an exemplary second intermediate image according to some embodiments of the present disclosure. FIG. 4C is a schematic diagram illustrating an exemplary second intermediate image after patchified according to some embodiments of the present disclosure. FIG. 4D is a schematic diagram illustrating exemplary image patches according to some embodiments of the present disclosure. As illustrated in FIGS. 4A-4D, a circular shift (e.g., certain pixels along a positive direction of a horizontal direction (or dimension) and certain pixels along a negative direction of a vertical direction) may be performed on the first intermediate image to generate the second intermediate image. Subsequently, the second intermediate image is patchified and divided into 16 non-overlapping image patches.

By uniformly dividing the second intermediate image into the image patches which are not overlapped, the division and subsequent operations can be simplified, thereby improving the efficiency of the generation of the second medical image.

In 3256, the processing device 140 (e.g., the generation module 220) may generate a third intermediate image corresponding to the current time step 325 based on the image patches, the shift vector, and a trained conditional diffusion model.

The third intermediate image corresponding to the current time step (also referred to as a current third intermediate image) refers to an image after processing generated in the current time step 325.

The trained conditional diffusion model (also referred to a conditional diffusion model) is a trained machine learning model, which can generate a processed image patch by processing each of the image patches. The processed image patch may have a higher image quality than the image patch. For example, each of the image patches may be input into the trained conditional diffusion model separately, and the trained conditional diffusion model may output the processed image patch corresponding to the image patch. In some embodiments, each of the image patches may also be referred to as a conditional image. The conditional image refers to prior information given to the trained conditional diffusion model, so as to restrict the output of the conditional diffusion model. For example, if the input is an image patch of a CT image, the output may also be an image patch of a CT image.

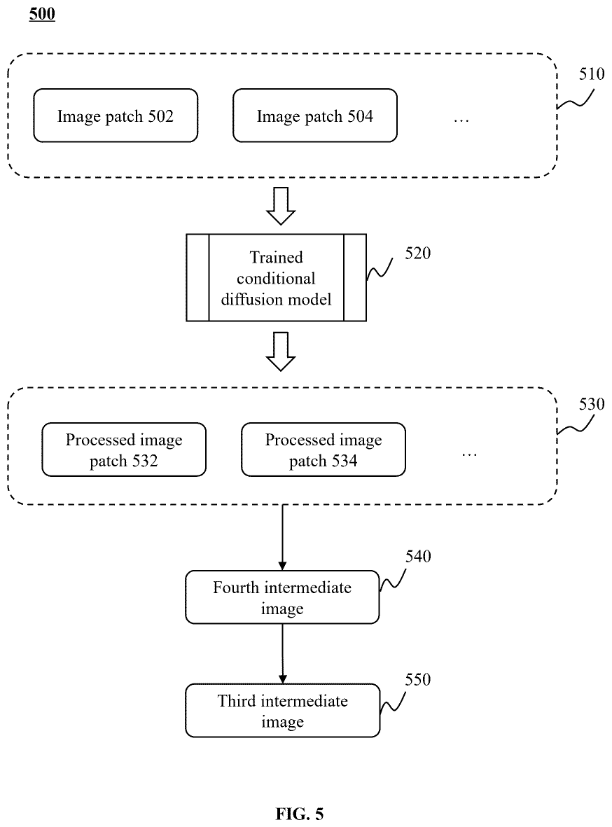

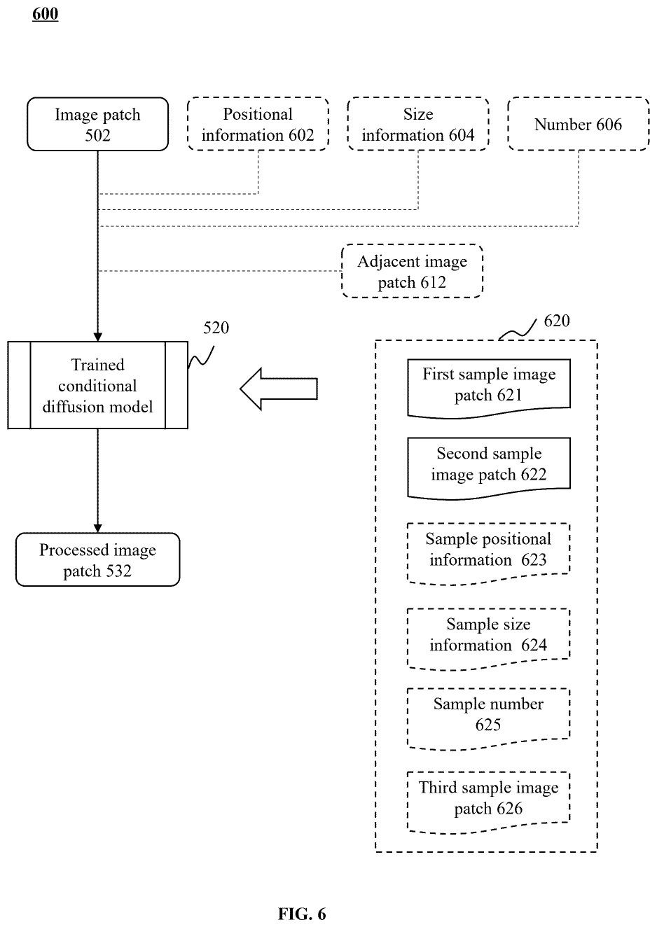

The processing device 140 may further generate a fourth intermediate image by splicing the processed image patches, and generate the third intermediate image corresponding to the current time step 325 by performing, based on the shift vector, a reverse circular shift on the fourth intermediate image. More descriptions regarding the generation of the processed image patch using the trained conditional diffusion model and the third intermediate image may be found elsewhere in the present disclosure. See, e.g., FIGS. 5-10 and relevant descriptions thereof.

In 3258, the processing device 140 (e.g., the generation module 220) may determine whether the third intermediate image satisfies a preset condition.

The preset condition may include a count condition, a quality condition, or the like, or any combination thereof.

The count condition may be that a preset count of time steps 320 has been completed. In some embodiments, the preset count of time steps 320 may be determined based on the count and/or the patch size of the image patches. For example, the larger the count (or the smaller the patch size) of the image patches, the larger the preset count of the time steps 320, so that the blocking artifacts caused by the image patches can be reduced or eliminated. In some embodiments, the preset count of time steps 320 may be determined based on the system default setting or set manually by the user. For example, the preset count of the time steps 320 may be a positive integer larger than 1, such as, 2, 5, 10, 20, 50, 100, 200, 500, 1000, etc. In some embodiments, the preset count is equal to or greater than 20, thereby ensuring the block artifact removing effect. The blocking artifacts refer to artifacts between adjacent processed image patches (e.g., obvious jumps or color differences at boundaries of the adjacent processed image patches) after the processed image patches are spliced together.

The quality condition may include that no artifacts exist in the third intermediate image or a degree of the artifacts is less than a degree threshold, a resolution of the third intermediate image exceeds a resolution threshold, or the like, or any combination thereof. The thresholds may be determined based on the system default setting or set manually by the user.

In some embodiments, the processing device 140 may determine whether the preset condition is satisfied by determining whether the third intermediate image satisfies the count condition. For example, the processing device 140 may determine whether the preset count of time steps 320 has been completed. If the preset count of time steps 320 has been completed, the processing device 140 may determine that the preset condition is satisfied and designate the third intermediate image as the second medical image. If the preset count of time steps 320 has not been completed, the processing device 140 may determine that the preset condition is not satisfied and proceed to a next time step 328.

In some embodiments, the processing device 140 may determine whether the preset condition is satisfied by evaluating whether the third intermediate image satisfies the quality condition. For example, the processing device 140 may determine whether a degree of the artifacts in the third intermediate image is less than the degree threshold. If the degree of the artifacts is less than the degree threshold, the processing device 140 may determine that the preset condition is satisfied and designate the third intermediate image as the second medical image. If the degree of the artifacts is not less than the degree threshold, the processing device 140 may determine that the preset condition is not satisfied and proceed to the next time step 328.

In some embodiments, the processing device 140 may determine whether the third intermediate image satisfies the count condition at first, and then determine whether the third intermediate image satisfies the quality condition. For example, the processing device 140 may firstly determine whether the preset count of time steps 320 has been completed. If the preset count of time steps 320 has not been completed, the processing device 140 may proceed to a next time step 328. If the preset count of time steps 320 has been completed, the processing device 140 may evaluate whether the third intermediate image satisfies the quality condition. If the third intermediate image satisfies the quality condition, the processing device 140 may designate the third intermediate image as the second medical image. If the third intermediate image does not satisfy the quality condition, the processing device 140 may proceed to the next time step 328.

In some embodiments, a post-processing operation may be performed on the third intermediate image for further eliminating the blocking artifacts. For example, if the preset count of the time steps 320 is less than a count threshold (e.g., 20, 30, 50, etc.) and/or the third intermediate image does not satisfy the quality condition, the processing device 140 may perform the post-processing operation on the third intermediate image. Alternatively, the processing device 140 may directly proceed to the next time step 328.

By determining whether the third intermediate image satisfies the count condition and the quality condition successively, different dimensions can be used to evaluate the third intermediate image, thereby improving the accuracy of the generation of the second medical image. Currently, a large number of time steps need to be performed when applying conditional diffusion models to yield accurate result, which is time-consuming. To reduce the processing time, the present disclosure introduces the quality condition. Since the quality condition needs to be satisfied, the preset count of time steps can first be set to a smaller value (e.g., the preset count of time steps can be smaller than the count threshold). If the evaluation result indicates that significant artifacts remain in the third intermediate image of the current time step, an additional time step or post-processing can be performed, thereby reducing the computational cost and improving the efficiency of the generation of the second medical image.

According to some embodiments of the present disclosure, the image patches instead of the entire first medical image are input into the conditional diffusion model, therefore, the computational resources required for the application of the conditional diffusion model can be significantly reduced, thereby enhancing computational efficiency. However, patch-based processing can introduce blocking artifacts (as the image patches are processed independently, the results from adjacent patches need to be stitched together, leading to noticeable discontinuities, color discrepancies, or visible block-like structures along patch boundaries). To mitigate blocking artifacts, the present disclosure applies a circular shift with a randomly generated shift vector to the first intermediate image at each timestep before partitioning it into image patches and feeding them into the conditional diffusion model. Due to the randomized shift magnitude introduced by the circular shift operation, the positions of edges and boundaries within the first medical image vary across the time steps. This approach effectively eliminates blocking artifacts in the resulting second medical image. In addition, the image patches are not overlapped, which can simplify the training and reference of the conditional diffusion model, thereby improving the efficiency of the application of the conditional diffusion model.

FIG. 5 is a schematic diagram illustrating an exemplary process 500 for generating a third intermediate image corresponding to a current time step according to some embodiments of the present disclosure.

As illustrated in FIG. 5, the processing device 140 (e.g., the generation module 220) may obtain image patches 510 (e.g., an image patch 502, an image patch 504, etc.). The image patches 510 may be obtained by dividing a second intermediate image corresponding to the current time step. More descriptions regarding the obtaining the image patches may be found elsewhere in the present disclosure. See, e.g., operation 3254 and relevant descriptions thereof.

In some embodiments, the processing device 140 may generate processed image patches 530 by inputting the image patches 510 into a trained conditional diffusion model 520, respectively. For example, the image patch 502 may be input into the trained conditional diffusion model 520, and the trained conditional diffusion model 520 may output a processed image patch 532. As another example, the image patch 504 may be input into the trained conditional diffusion model 520, and the trained conditional diffusion model 520 may output a processed image patch 534.

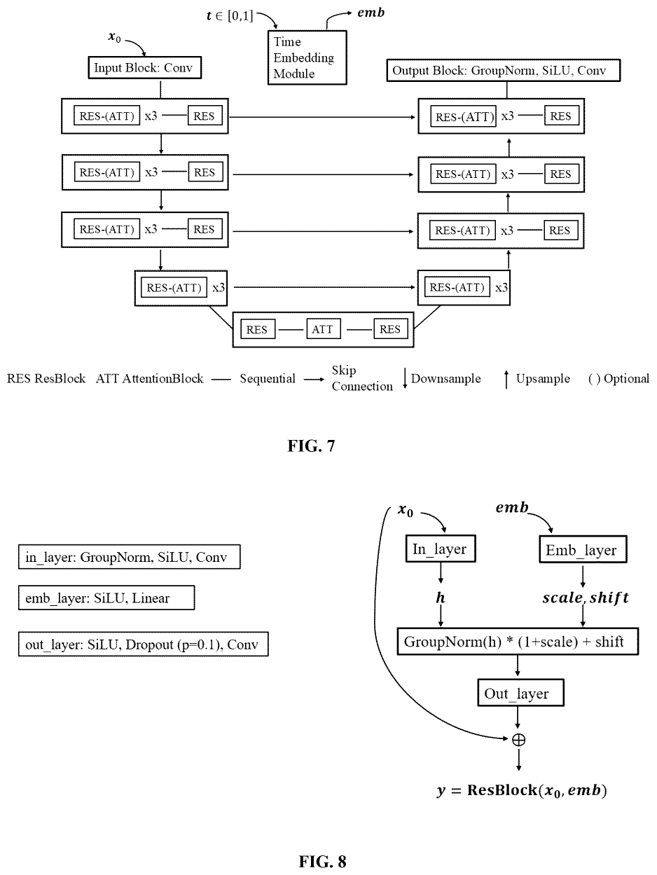

The trained conditional diffusion model 520 may be a trained machine learning model configured to determine the processed image patch based on the image patch. More descriptions regarding the trained conditional diffusion model and the generation of the processed image patch may be found elsewhere in the present disclosure. See, e.g., FIGS. 6-10 and relevant descriptions thereof.

In some embodiments, the processing device 140 may generate a fourth intermediate image 540 by splicing the processed image patches 530. For example, the processing device 140 may directly splice adjacent processed image patches 530 to generate the fourth intermediate image 540. As another example, the processing device 140 may splice the processed image patches 530 based on corresponding positions of the processed image patches 530 in the second intermediate image corresponding to the current time step.

In some embodiments, the processing device 140 may generate a third intermediate image 550 corresponding to the current time step by performing, based on the shift vector, a reverse circular shift on the fourth intermediate image 540.

The shift vector is used to perform the circular shift on the first intermediate image of the current time step to generate the second intermediate image corresponding to the current time step. More descriptions regarding the shift vector may be found elsewhere in the present disclosure. See, e.g., operation 3252 and relevant descriptions thereof.

The reverse circular shift may be a reverse process with respect to the circular shift operation performed on the first intermediate image. As for the example described in connection with operation 3252 in which the first element and the second element of the shift vector are +3 and −2, respectively, the reverse circular shift may be performed such that each pixel in the fourth intermediate image 540 is shifted three pixels along the negative direction of the horizontal dimension and two pixels along the positive direction of the vertical dimension to generate the third intermediate image 550.

FIG. 6 is a schematic diagram illustrating an exemplary process 600 for generating a processed image patch through a trained conditional diffusion model according to some embodiments of the present disclosure.

For illustration purposes, the generation of the processed image patch 532 is taken as an example and described below.

As illustrated in FIG. 6, the image patch 502 may be input into the trained conditional diffusion model 520, and the trained conditional diffusion model 520 may output the processed image patch 532.

In some embodiments, the trained conditional diffusion model 520 may be obtained by training an initial conditional diffusion model using training samples 620. In some embodiments, each of the training samples 620 may include a first sample image patch 621 as an input of the initial conditional diffusion model and a corresponding second sample image patch 622 as a label of the initial conditional diffusion model.

In some embodiments, the processing device 140 may obtain a first sample image and a second sample image of a sample subject. The processing device 140 may generate a first shifted sample image by performing, based on a randomly generated sample shift vector, a circular shift on the first sample image, and generate a second shifted sample image by performing, based on the sample shift vector, a circular shift on the second sample image. Further, the processing device 140 may extract the first sample image patch 621 from the first shifted sample image and the corresponding second sample image patch 622 from the second shifted sample image.

The first sample image and the second sample image (also referred to as a pair of sample images) may be corresponding medical images of the sample subject. For example, the first sample image and the second sample image may be acquired using X-ray imaging. In some embodiments, the second sample image may have a higher image quality than the first sample image. For example, the second sample image may include less artifacts (e.g., motion artifacts) than the first sample image. As another example, the second sample image may correspond to a higher radiation dose than the first sample image. As still another example, the second sample image may correspond to a higher sampling rate than the first sample image. As yet another example, the second sample image may correspond to a longer scanning time than the first sample image. Merely by way of example, the first sample image may be a simulated artifact-corrupted image of sample blood vessels corresponding to a middle motion phase among multiple motion phases, and the second sample image may be a simulated artifact-free image of the sample blood vessels corresponding to the middle motion phase as described in FIG. 11.

In some embodiments, the processing device 140 may obtain the first sample image and the second sample image from an imaging device (e.g., the imaging device 110) or a storage device (e.g., the storage device 150, a database, or an external storage) that stores the first sample image and the second sample image.

The generation of the first shifted sample image and/or the second shifted sample image may be similar to the generation of the second intermediate image described in operation 3252. Taking the first shifted sample image as an example, the processing device 140 may generate the first shifted sample image by concatenating the first sample image with at least one of a random noise image or a positional coordinate array corresponding to the first sample image, and performing the circular shift on the concatenated image based on the randomly generated sample shift vector.

The first sample image patch 621 and the corresponding second sample image patch 622 (also referred to as a pair of sample image patches) may correspond to a same portion of the sample subject. For example, the processing device 140 may extract the first sample image patch 621 from the first shifted sample image and the corresponding second sample image patch 622 from the second shifted sample image based on the same portion of the sample subject (e.g., a region of interest). As another example, the processing device 140 may perform a registration on the first shifted sample image and the second shifted sample image, and extract the first sample image patch 621 and the corresponding second sample image patch 622 from the registered first shifted sample image and the registered second shifted sample image, respectively. For instance, the processing device 140 may patchify the registered first shifted sample image and the registered second shifted sample image into a same count of image patches, and determine at least one pair of sample image patches from the image patches.

In some embodiments, the initial conditional diffusion model may be trained according to a machine learning algorithm. For example, the processing device 140 may generate the trained conditional diffusion model 520 according to a supervised machine learning algorithm by performing one or more iterations to iteratively update model parameter(s) of the initial conditional diffusion model.

Merely by way of example, the training of the initial conditional diffusion model may include an iterative process. The training samples 620 may be used to iteratively update model parameter(s) of the initial conditional diffusion model until a termination condition is satisfied. Exemplary termination conditions may include that a value of a loss function corresponding to the initial conditional diffusion model is below a threshold value, a difference of values of the loss function obtained in a previous iteration and the current iteration is within a difference threshold value, a certain count of iterations has been performed, etc.

For instance, in a current iteration, a first sample image patch 621 of a training sample may be input into the initial conditional diffusion model, and the initial conditional diffusion model may generate a predicted image patch corresponding to the first sample image patch 621. Then, a value of the loss function (e.g., also referred to as a first loss function) may be determined to measure a difference between the predicted image patch and the training label (e.g., a corresponding second sample image patch 622 in the training sample). If it is determined that the termination condition is satisfied in the current iteration, the initial conditional diffusion model may be designated as the trained conditional diffusion model 520; otherwise, the initial conditional diffusion model may be further updated based on the value of the loss function.

In some embodiments, the first sample image and the second sample image may be acquired using the X-ray imaging, and the first loss function may include a first loss term and a second loss term. The first loss term may relate to a difference between the predicted image patch output by the initial conditional diffusion model and the second sample image patch, and be configured to constrain the difference between the predicted image patch and the training label (e.g., the corresponding second sample image patch 622). By introducing the first loss term, the accuracy of local details in the predicted image patch can be ensured.

The second loss term may relate to a difference between back-projection data of the predicted image patch and back-projection data of the first sample image patch 621, and be configured to constrain a difference between the back-projection data of the predicted image patch and the back-projection data of the first sample image patch 621. By introducing the second loss term, the rationality of the global structure of the predicted image patch can be ensured.

In some embodiments, the first loss term may correspond to a first weight, and the second loss term may correspond to a second weight. If the first weight is relatively larger than the second weight during the training, i.e., the training relies on the first loss term, the noise would be overfit. If the second weight is relatively larger than the first weight during the training, i.e., the training relies on the second loss term, high-frequency details (or details of edge regions of the first sample image patch 621) would be missed. Therefore, the first loss term and the second loss term (or the first weight and the second weight) may be compromised, thereby avoiding the overfitting of the noise and the loss of the high-frequency details. For example, a difference between the first weight and the second weight may be less than a difference threshold, such as, a range from −0.1 to 0.1, a range from −0.2 to 0.2, a range from −0.3 to 0.3, etc. As another example, a ratio of the first weight to the second weight may be less than a ratio threshold, such as, a range from 0.5 to 2, a range from 0.3 to 3, a range from 0.25 to 4, a range from 0.2 to 5, etc.

As still another example, the first weight and the second weight may be dynamically adjusted during the training. For instance, the first weight and the second weight in the current iteration may be determined based on a ratio of a count of iterations that have been completed to a preset count of iterations. For example, the first weight may negatively correlate with the ratio, and the second weight may positively correlate with the ratio. As another example, when the ratio is less than 0.5, the first weight may be larger than the second weight. Otherwise, the first weight may be smaller than the second weight. Merely by way of example, the processing device 140 may divide the training into a first stage (also referred to an early stage), a second stage (also referred to a medium stage), and a third stage (also referred to a final stage) based on the ratio of the count of iterations that have been completed to the preset count of iterations, wherein the first stage corresponds to a first ratio range from 0 to 1/3, the second stage corresponds to a second ratio range from 1/3 to 2/3, and the third stage corresponds to a third ratio range from 2/3 to 1. Correspondingly, when the ratio is within the first ratio range, the first weight may be 0.8, and the second weight may be 0.2. When the ratio is within the second ratio range, the first weight and the second weight may be 0.5. When the ratio is within the third ratio range, the first weight may be 0.2, and the second weight may be 0.8.

By dynamically adjusting the first weight and the second weight, the local details in the predicted image patch can be quickly converged in the early stage, and the rationality of the global structure of the predicted image patch can be ensured in the final stage. This can improve the efficiency and accuracy of the generation of the predicted image patch, thereby improving the efficiency and accuracy of the model training.

In some embodiments, the training of the initial conditional diffusion model may include a forward diffusion process (e.g., a noising process) and a reverse diffusion process (e.g., a denoising process). The trained conditional diffusion model may represent the distribution of the training sample by reversing the forward diffusion process. The forward diffusion process gradually adds a sample noise (e.g., Gaussian noise) to an input (e.g., the first sample image patch 621) over continuous time or discrete iterations, and generates a noisy image patch based on the input. The reverse diffusion process gradually denoises the noisy image patch. With a sufficient count of iterations, the reverse diffusion process is also close to a Gaussian distribution and can be represented by the initial conditional diffusion model. That is, a predicted noise is determined (e.g., output by the initial conditional diffusion model).

A second loss function (e.g., a mean square deviation between the predicted noise and a ground truth noise (i.e., the sample noise)) corresponding to the initial conditional diffusion model may be determined. Further, the trained conditional diffusion model may be obtained by updating the model parameter(s) of the initial conditional diffusion model until the second loss function is minimized.

Merely by way of example, the forward diffusion process (e.g., the noising process) may be expressed as Equation (1):

$$q ( {z}_{t} \vert x ) = 𝒩 ( {z}_{t} ; {α}_{t} x , {σ}_{t}^{2} I ) , \qquad ( 1 )$$

where x represents the input (e.g., the first sample image patch 621), t represents a time step, which is within a range from 0 to 1, I represents an identity matrix, zₜ is a latent variable of the input x, and αₜ, σₜ∈[0,1] are hyperparameters for a noise schedule. zₜ may be regarded as an immediate image (e.g., the noisy image patch) after the input x is added with sample noise.

When only a variance-preserving cosine noise schedule is considered,

$${α}_{t}^{2} + {σ}_{t}^{2} = 1 ,$$

and αₜ=cos(πt/2).

In some embodiments, the sample noise and the input x are linearly related in the diffusion process, as Equation (2):

zₜ=αₜx+σₜϵₜ, (2)

where ϵₜ represents the ground truth noise, and ϵₜ~ [symbol] (0,I).

The reverse diffusion process p(zₛ|zₜ), 0≤s<t≤1, is a process to generate a denoised image patch zₛ from zₜ and may be represented by the initial conditional diffusion model as Equation (3):

p(zₛ|zₜ)= [symbol] (zₛ;μ_{θ}(zₜ;s,t),σ²(s,t)I), (3)

where μ_{θ}(⋅) represents the predicted image patch with parameters θ corresponding to the input x, αₛ, σₛ, αₜ, σₜ∈[0,1] are hyperparameters for the noise schedule, and σ²(s,t) is a scalar function of αₜ, αₛ, σₜ and σₛ.

The initial conditional diffusion model may also be used to predict noise. For example, the predicted noise may be output by the initial conditional diffusion model and be denoted by ϵₐ(⋅). The second loss function may be constructed based on the predicted noise ϵ_{θ}(⋅) and the ground truth noise ϵₜ. For example, the second loss function may be represented by Equation (4) below:

Lₛᵢₘₚₗₑ:= [symbol] [∥ϵₜ−ϵ_{θ}(zₜ,s,t)∥]. (4)

The second loss function may be used to update the initial conditional diffusion model in a similar manner as how the first loss function is used to update the initial conditional diffusion model. In some embodiments, the first loss function and the second loss function may be simultaneously used to train the initial conditional diffusion model. For example, the first loss function may correspond to a third weight, and the second loss function may correspond to a fourth weight. The third weight and the fourth weight may be determined based on a system default setting or set manually by a user. In some embodiments, one of the first loss function and the second loss function may be used to train the initial conditional diffusion model. For example, the first loss function is used to train the initial conditional diffusion model, and the second loss function is used to verify the trained conditional diffusion model.

In some embodiments, an alternative parameterization (e.g., ν-prediction) may be adopted for better numerical stability.

In some embodiments, the trained conditional diffusion model 520 may also be obtained according to other training manners. For example, the trained conditional diffusion model 520 may be obtained based on an initial learning rate (e.g., 0.1) and/or an attenuation strategy using the training samples 620.

It should be noted that the trained conditional diffusion model 520 is merely provided for illustration purposes, and can be modified according to an actual need. For example, the trained conditional diffusion model 520 may include multiple input channels, wherein one of the input channels is configured to input an image patch (e.g., the image patch 502), and the other channel(s) are configured to input other information. The other information may include positional information 602 of the image patch 502, size information 604 of the image patch 502, a number 606 of a current time step, one or more adjacent image patches 612 corresponding to the image patch 502, or the like, or any combination thereof. In some embodiments, each input channel may correspond to a type of information. That is, each of the input channels may correspond to one of the image patch 502, the positional information 602, the size information 604, the number 606, and one of the one or more adjacent image patches 612.

The positional information 602 refers to information regarding a position of the image patch 502 in the second intermediate image. For example, the positional information 602 of the image patch 502 may include coordinates of a center point, a corner point, an edge, etc., of the image patch 502 in the second intermediate image.

In some embodiments, the positional information 602 may be determined when the image patch 502 is determined from the second intermediate image. For example, the positional information 602 of the image patch 502 may be input into the trained conditional diffusion model 520 with the image patch 502 to generate the processed image patch 532.

Correspondingly, each of the training samples 620 may further include sample positional information 623 of the training sample. The sample positional information 623 may be obtained in a similar manner as how the positional information 602 is obtained.

The size information 604 may be a patch size of the image patch 502. More descriptions regarding the patch size may be found elsewhere in the present disclosure. See, e.g., operation 3254 and relevant descriptions thereof. For example, the size information 604 of the image patch 502 may be input into the trained conditional diffusion model 520 with the image patch 502 to generate the processed image patch 532.

Correspondingly, each of the training samples 620 may further include sample size information 624 of the training sample. The sample size information 624 may be obtained in a similar manner as how the size information 604 is obtained.

The number 606 of the current time step may also be input into the trained conditional diffusion model 520. Correspondingly, each of the training samples 620 may further include sample number 625 corresponding to the training sample.

In some embodiments, the one or more adjacent image patches 612 corresponding to the image patch 502 may be determined from one or more adjacent medical images of a first medical image. For example, if the first medical image is a 2D image (or slice) corresponding to an anatomical plane (e.g., a transverse section, a coronal section, or a cross section), the one or more adjacent medical images may correspond to one or more neighboring anatomical planes of the anatomical plane. For instance, if the first medical image corresponds to the transverse section, the one or more adjacent medical images may correspond to adjacent transverse sections of the transverse section. Merely by way of example, if the processing device 140 obtains a 3D image including a plurality of 2D images (or slices), and the first medical image is a fifth slice, the one or more adjacent medical images may be at least one of a fourth slice (i.e., a previous slice) or a sixth slice (i.e., a next slice). In some embodiments, each of the one or more adjacent image patches 612 may be determined from the corresponding adjacent medical image in a similar manner as how the image patch is determined from the first medical image as described in FIG. 3.

In some embodiments, the one or more adjacent image patches 612 corresponding to the image patch 502 may be input into the trained conditional diffusion model 520 with the image patch 502 (and optionally with at least one of the positional information 602, the size information 604, or the number 606) to generate the processed image patch 532. For example, the image patch 502 corresponding to the fifth slice, an image patch corresponding to the fourth slice, and an image patch corresponding to the sixth slice may be input into the trained conditional diffusion model 520 (e.g., three input channels of the trained conditional diffusion model 520), and the trained conditional diffusion model 520 may output the processed image patch 532. For instance, the image patch 502 corresponding to the fifth slice, the image patch corresponding to the fourth slice, and the image patch corresponding to the sixth slice may be spliced to form a 2.5D input. At this time, the trained conditional diffusion model 520 may also referred to as a 2.5D conditional diffusion model. As another example, the image patch 502 corresponding to the fifth slice, the image patch corresponding to the fourth slice, the image patch corresponding to the sixth slice, and a random noise patch (from the random noise image) may be input into the trained conditional diffusion model 520 (e.g., four input channels of the trained conditional diffusion model 520), and the trained conditional diffusion model 520 may output the processed image patch 532. By inputting the random noise patch with the image patches, robustness of the trained conditional diffusion model 520 can be enhanced, and diversity generated by the trained conditional diffusion model 520 can be improved.

Correspondingly, each of the training samples 620 may further include one or more third sample image patches 626 of the training sample. The one or more third sample image patches 626 may be obtained in a similar manner as how the one or more adjacent image patches 612 are obtained.

By introducing at least one of the positional information, the size information, the number, or the one or more adjacent image patches, more reference information can be used to generate the processed image patch (e.g., the trained conditional diffusion model can learn a positional relationship between the first sample image patch 621 and the first sample image), thereby improving the accuracy of the generation of the processed image patch.

Furthermore, the 2.5D conditional diffusion model can be provided by concatenating the one or more adjacent image patches and/or the random noise patch as multi-channel inputs (e.g., 3-channel inputs, 5-channel inputs, etc.) of the multiple input channels of the trained conditional diffusion model, which achieves a balance of performance and computational burdens. In addition, although the underlying network is still two-dimensional, the 2.5D conditional diffusion model can leverage through-plane corrections, thereby improving the application range of the trained conditional diffusion model.

In some embodiments, a weight value may be designated to each of the multiple input channels of the trained conditional diffusion model 520, thereby adjusting influences of different input channels on the generation of the processed image patch 532 through the trained conditional diffusion model 520. This can facilitate sharing and integration of information between the input channels, thereby improving the accuracy of the generation of the processed image patch 532.

In some embodiments, the input of the trained conditional diffusion model 520 may be altered. For example, the input may be other data generated based on the image patch 502.

For instance, the processing device 140 may generate 180° parallel-beam sinogram data by performing forward projection on the image patch 502, divide the 180° parallel-beam sinogram data into multiple sinogram datasets corresponding to different partial angular ranges, and generate partial angle reconstruction (PAR) image blocks by performing back-projection on the sinogram datasets. Then, the PAR image blocks may be input into the trained conditional diffusion model 520 to generate the processed image patch 532. Correspondingly, each of the training samples 620 may include sample PAR image blocks corresponding to the first sample image patch 621.

As another example, the processing device 140 may input the PAR image blocks into the trained conditional diffusion model 520 with the image patch 502 to generate the processed image patch 532. Correspondingly, each of the training samples 620 may include the first sample image patch 621 and the sample PAR image blocks corresponding to the first sample image patch 621. More descriptions regarding the generation of the processed image patch based on the PAR image blocks may be found elsewhere in the present disclosure. See, e.g., FIG. 9 and relevant descriptions thereof.

As still another example, the initial diffusion model may be trained through the training dataset at an image-level. That is, the first sample image and the second sample image may be used to train the initial conditional diffusion model.

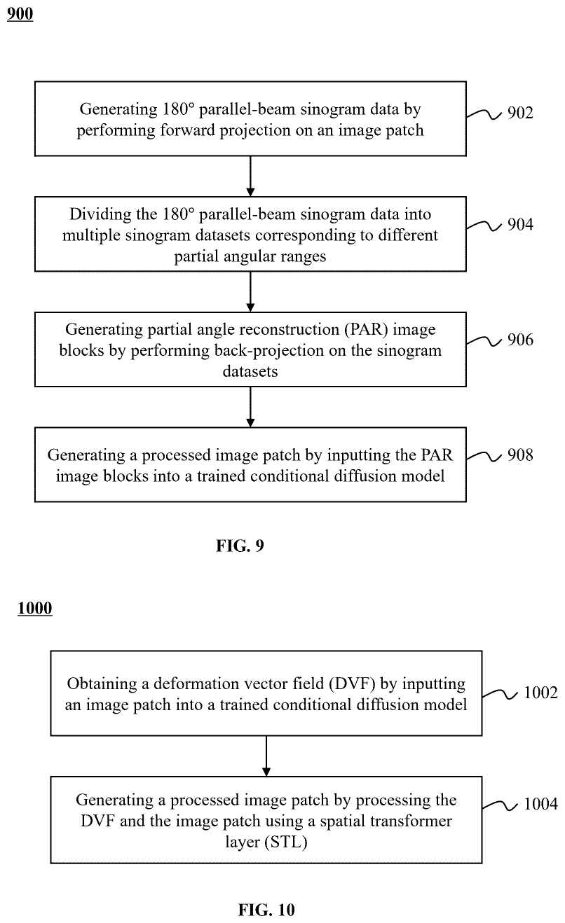

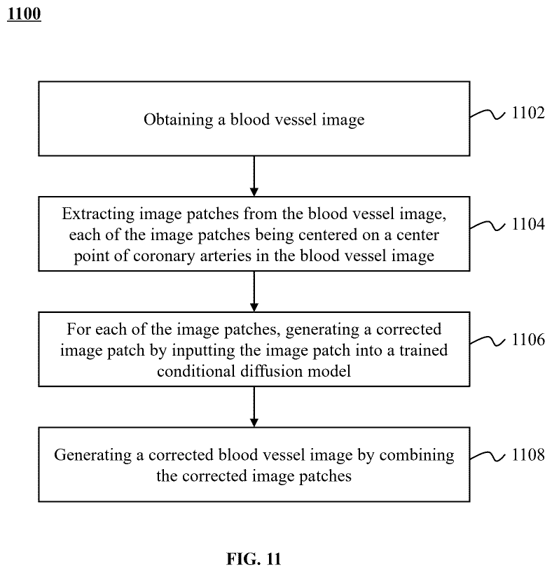

In some embodiments, an architecture of the trained conditional diffusion model may also be altered. For example, the trained conditional diffusion model may be connected with a spatial transformer layer (STL). Therefore, the trained conditional diffusion model 520 may be trained end-to-end within a supervised learning framework that incorporates the initial conditional diffusion model and the STL. For instance, the processing device 140 may obtain a deformation vector field (DVF) by inputting the image patch 502 into the trained conditional diffusion model 520, and generate the processed image patch 532 by processing the DVF and the image patch 502 using the STL. As another example, the processing device 140 may input the PAR image blocks (and optionally the image patch 502) into the trained conditional diffusion model 520 to generate the DVF, and generate the processed image patch 532 by processing the DVF and the image patch 502 using the STL. More descriptions regarding the generation of the processed image patch based on the DVF and the image patch may be found elsewhere in the present disclosure. See, e.g., FIG. 10 and relevant descriptions thereof.

For another example, the trained conditional diffusion model may be incorporated with attention modules. For instance, a self-attention module may be added at a lowest resolution branch of the trained conditional diffusion model (e.g., a denoising diffusion probability model (DDPM)). As another example, as illustrated in FIG. 7, attention layers (denoted as “ATT” in FIG. 7) may be appended to residual blocks (denoted as “RES” in FIG. 7) of the trained conditional diffusion model, such as, in higher-resolution branches.

In some embodiments, the residual blocks of the trained conditional diffusion model may further include time embeddings. For example, as illustrated in FIG. 8, a residual block may include an input layer (denoted as “In_layer”), a time embedding layer (denoted as “Emb_layer”), a residual layer, and an output layer (denoted as “Out layer”). The time embedding layer may be configured to scale and shift intermediate features before the output layer. For example, the intermediate features may include a feature corresponding to the time step.

By altering the trained conditional diffusion model, an application range of the trained conditional diffusion model can be improved. Furthermore, different functions can be introduced to the trained conditional diffusion model, thereby improving the accuracy of the trained conditional diffusion model.

FIG. 9 is a flowchart illustrating an exemplary process 900 for generating a processed image patch according to some embodiments of the present disclosure.

In 902, the processing device 140 (e.g., the generation module 220) may generate 180° parallel-beam sinogram data by performing forward projection on an image patch.

The forward projection refers to a mathematical operation simulating a physical process of CT scanning. For example, the forward projection may include assuming X-rays passing through the image patch, and determining a sum (e.g., a line integral) of pixel values of the image patch along each path of the x-rays. This process can simulate X-ray attenuation signals received by a detector of an imaging device at different angles.

The sinogram data refers to original projection data of the CT scanning. In some embodiments, the sinogram data is a 2D array, wherein one dimension of the 2D array represents a channel position of the detector, and the other dimension represents a projection angle (or a rotation angle). Each point in the sinogram data represents a projection value (total attenuation) received at a specific angle and channel position of the detector.

The 180° parallel-beam sinogram data refers to projection data obtained by projecting the image patch with dense parallel beams within a range from 0° to 180° (or an equivalent 180° range). For example, the forward projection may be performed on the image patch with the dense parallel beams within the range from 0° to 180° to generate the 180° parallel-beam sinogram data.

In 904, the processing device 140 (e.g., the generation module 220) may divide the 180° parallel-beam sinogram data into multiple sinogram datasets corresponding to different partial angular ranges.

In some embodiments, the range from 0° to 180° may be divided into a plurality of partial angular ranges. For example, the plurality of partial angular ranges may not be overlapped. For instance, the range from 0° to 180° may be divided into four partial angular ranges, including 0° to 45°, 450 to 90°, 900 to 135°, and 1350 to 180°. As another example, the range from 0° to 180° may be divided into six partial angular ranges, including 0° to 30°, 300 to 60°, 600 to 90°, 900 to 120°, 1200 to 150°, and 1500 to 180°.

As another example, a portion of the plurality of partial angular ranges may be overlapped. For instance, the range from 0° to 180° may be divided into four partial angular ranges, including 0° to 60°, 450 to 105°, 900 to 150°, and 1200 to 180°.

A sinogram dataset refers to a sub-set (a portion) of the 180° parallel-beam sinogram data. Each of the sinogram datasets may include projection data rows belonging to a corresponding partial angular range in the 180° parallel-beam sinogram data, and each row may correspond to the projection data at one angle.

In some embodiments, the processing device 140 may determine a count of the plurality of partial angular ranges based on actual scanning requirements. For example, the larger span of each partial angular range (or the less the count of the plurality of angular ranges, or the larger the overlapped range), the less the temporal resolution, but the higher the reconstruction quality. Therefore, a tradeoff between the temporal resolution and the reconstruction quality may be determined according to the actual scanning requirements.

In 906, the processing device 140 (e.g., the generation module 220) may generate partial angle reconstruction (PAR) image blocks by performing back-projection on the sinogram datasets.

In some embodiments, the processing device 140 may perform back-projection on each of the sinogram datasets using a back-projection algorithm. Exemplary back-projection algorithms may include a filtering back-projection (FBP) algorithm, a simple back-projection algorithm, or the like, or any combination thereof.

Each PAR image block may correspond to one of the sinogram datasets. That is, each PAR image block may correspond to one of the plurality of partial angular ranges. Therefore, the quality of each PAR image block may be relatively low. For example, each PAR image block may include streak artifacts (stripes) and have poor resolution. However, since motion artifacts in the image patch are angle-dependent (e.g., the motion phases are different at different time points), each PAR image block may reflect a specific motion phase corresponding to its partial angular range during the data acquisition (e.g., the CT scanning), and thus is less corrupted by physiological motion.

In 908, the processing device 140 (e.g., the generation module 220) may generate a processed image patch by inputting the PAR image blocks into a trained conditional diffusion model.

For example, the processing device 140 may input the PAR image blocks into a trained conditional diffusion model (e.g., the trained conditional diffusion model 520), and the trained conditional diffusion model may output the processed image patch. As another example, the processing device 140 may input the PAR image blocks and the image patch into the trained conditional diffusion model (e.g., the trained conditional diffusion model 520), and the trained conditional diffusion model may output the processed image patch.

By obtaining the PAR image blocks, the temporal resolution can be improved. Further, different PAR image blocks can correspond to different motion phases of the physiological motion (e.g., cardiac motion) of the subject, which can input more motion information (e.g., the motion phases) into the trained conditional diffusion model, thereby improving the accuracy and the image quality of the processed image patch (e.g., motion artifact correction).

In addition, in an actual data acquisition (e.g., the CT scanning), complete sinogram data corresponding to the range from 0° to 180° may not be obtained due to the physiological motion or time limitation. The simulation through the PAR image blocks can improve the suitability between the trained conditional diffusion model and a short scan requirement, thereby further improving the accuracy and the image quality of the processed image patch.

FIG. 10 is a flowchart illustrating an exemplary process 1000 for generating a processed image patch according to some embodiments of the present disclosure.

In 1002, the processing device 140 (e.g., the generation module 220) may obtain a deformation vector field (DVF) by inputting an image patch into a trained conditional diffusion model.

The DVF refers to a deformation field between the image patch and a processed image patch.

The trained conditional diffusion model is a trained machine learning model that generates the DVF by processing the image patch, and such model is also referred to as a third conditional diffusion model. For example, the processing device 140 may input the image patch (and/or PAR image blocks corresponding to the image patch as described in FIG. 9) into the third conditional diffusion model, and the third conditional diffusion model may output the DVF.

In 1004, the processing device 140 (e.g., the generation module 220) may generate a processed image patch by processing the DVF and the image patch using a spatial transformer layer (STL).

The STL may be a differentiable module that is configured to perform geometric transformations, such as deformation, rotation, scaling, etc.

In some embodiments, the STL may be connected to the third conditional diffusion model. For example, the STL may be connected to a functional layer of an architecture of an initial conditional diffusion model as described in FIG. 6. As another example, the initial conditional diffusion model and the STL may be connected to form a supervised learning framework. Therefore, the STL may process the DVF output by the third conditional diffusion model and the image patch to generate the processed image patch.

In some embodiments, the third conditional diffusion model may be trained end-to-end within the supervised learning framework that incorporates the initial conditional diffusion model and the STL. For example, the third conditional diffusion model may be obtained by training the supervised learning framework using training samples (e.g., the training samples 620). For example, the training of the supervised learning framework that incorporates the initial conditional diffusion model and the STL may include an iterative process. The training samples 620 may be used to iteratively update model parameter(s) of the initial conditional diffusion model until a termination condition is satisfied.

For instance, in a current iteration, one first sample image patch 621 of a training sample may be input into the initial conditional diffusion model, and the initial conditional diffusion model may generate a predicted DVF corresponding to the first sample image patch 621. The STL may generate a predicted image patch by processing the predicted DVF and the first sample image patch 621. Then, a value of the loss function may be determined to measure a difference between the predicted image patch and the training label (e.g., a corresponding second sample image patch 622 in the training sample). If it is determined that the termination condition is satisfied in the current iteration, the initial conditional diffusion model may be designated as the third conditional diffusion model; otherwise, the initial conditional diffusion model may be further updated based on the value of the loss function.

In some embodiments, each of the training samples may include sample PAR image blocks corresponding to the first sample image patch 621. Correspondingly, in the current iteration, the sample PAR image blocks (and the first sample image patch 621) of the training sample may be input into the initial conditional diffusion model, and the initial conditional diffusion model may generate the predicted DVF corresponding to the first sample image patch 621.

By introducing the STL, the predicted DVF can be applied to the input image patch, which can achieve pixel-level geometric correction, and maintain end-to-end differentiability of the trained conditional diffusion model, thereby improving the accuracy of the trained conditional diffusion model.

FIG. 11 is a flowchart illustrating an exemplary process 1100 for generating a corrected blood vessel image according to some embodiments of the present disclosure. Process 1100 may be implemented in the imaging system 100 illustrated in FIG. 1. For example, the process 1100 may be stored in the storage device 150 in the form of instructions (e.g., an application), and invoked and/or executed by the processing device 140.

Cardiac computed tomography (CT) (e.g., coronary computed tomography angiography (CCTA)) has rapidly grown as a substitute for conventional invasive coronary angiography for the detection of significant stenosis (e.g., aortic stenosis, restenosis) and other coronary artery diseases (CAD). A lumen diameter of a normal coronary artery varies in a diameter range from 1.9 millimeters (mm) to 4.5 mm. Stents used in the coronary artery have a similar diameter range. Both the coronary artery and the stents can be resolved with modern high-resolution (0.2-0.5 mm) CT scanners in static.

However, the visualization of the coronary arteries presents challenges as the coronary arteries can be as small as 1 mm in lumen diameter, and undergo complex three-dimensional motion during cardiac cycles. For instance, the heart constantly cycles between systole and diastole, a cardiac cycle lasts about 0.8 seconds (s), but a typical gantry rotation time of a CT system is 0.2-0.3 s, during which the heart can move several millimeters. Therefore, an insufficient temporal resolution of the existing CT system results in motion artifacts, including blurring, shading, distortion, etc. In addition, the administration of a contrast agent and blood flow dynamics further complicates the imaging of the coronary arteries.

Conventionally, ECG-gated scan protocols aim to find the best phase with small heart motions. The best-phase selection itself is a challenging problem as the best phase can vary dramatically from patient to patient. More importantly, motion artifacts due to heart motion remain in the best-phase reconstructions. Iterative algorithms have been proposed to improve temporal resolution by formulating motion correction/compensation as an ill-posed reconstruction problem. Some conventional approaches split a short scan into two half short scans and reconstruct two limited angle data with a prior image constraint, achieving roughly doubled temporal resolution. However, the performance can be highly dependent on the prior image, a motion-corrupted reconstruction.

Recognizing the existence of unavoidable motions, motion estimation and motion compensation (ME/MC) methods were proposed based on image domain motion artifact metrics or partial angle reconstructions (PAR). PAR image blocks, as the name suggests, are reconstructed using several consecutive partial angles partitioned from a single short-scan acquisition. Each PAR image block has a higher temporal resolution despite its severe limited-angle artifacts. These PAR image blocks are used to derive a motion vector field (MVF) between two conjugate PARs that are 180 degrees apart. The estimated MVF is applied to all PAR image blocks to reconstruct any selected cardiac phase. Nevertheless, the conjugate pair of PAR image blocks is most sensitive to the motion perpendicular to the view direction yet holds no motion information for the parallel direction. Furthermore, small motions can not be well estimated and sometimes totally ignored due to the low-pass filtering nature of the motion estimation process.

Deep learning methods have emerged and have been utilized to reduce motion artifacts as image postprocessing or image-style transfer, or to predict cardiac motions (e.g., MVF) with convolutional neural networks or a spatial transformer module. Recently, conditional diffusion models have been shown to generate high-quality and high-resolution images from the distribution of a training dataset. With extra prior images, the conditional diffusion models can robustly generate images constrained/conditioned by the prior and be suitable for extensive medical imaging applications, such as image denoising, super-resolution, reconstruction, artifact reduction, etc.

Therefore, to use the conditional diffusion models to correct blood vessel images, such as the CCTA images, the process 1100 may be performed.

In 1102, the processing device 140 (e.g., the obtaining module 210) may obtain a blood vessel image.

The blood vessel image refers to an image including blood vessel(s) of a subject. Exemplary blood vessels may include arteries (e.g., coronal arteries, aortae, carotid arteries, brachial arteries, iliac arteries, femoral arteries, pulmonary arteries, etc.), veins (e.g., superior vena cava, inferior vena cava, carotid veins, great saphenous veins, hepatic portal veins, pulmonary veins, etc.), blood capillaries, or the like, or any combination thereof.

Exemplary blood vessel images may include an X-ray angiography image (e.g., a digital subtraction angiography (DSA) image), a computed tomography angiography (CTA) image (e.g., a coronary CT angiography (CCTA) image), a magnetic resonance angiography (MRA) image, an ultrasound angiography image (e.g., a Doppler ultrasound angiography image, such as, a color Doppler ultrasound angiography image, a spectral Doppler ultrasound angiography image, etc.), a nuclear medicine angiography image, an optical image (e.g., an optical coherence tomography angiography (OCTA) image, a laser speckle contrast imaging (LSCI) image, etc.), or the like, or any combination thereof.

In some embodiments, the processing device 140 may obtain the blood vessel image from an imaging device (e.g., the imaging device 110) or a storage device (e.g., the storage device 150, a database, or an external storage) that stores the blood vessel image. Alternatively, the processing device 140 may obtain the blood vessel image by reconstructing image data corresponding to the blood vessel image.

In some embodiments, the blood vessel image may be an exemplary embodiment of the first medical image 310 as described in FIG. 3.

In 1104, the processing device 140 (e.g., the generation module 220) may extract image patches from the blood vessel image.

An image patch may be a small region extracted from the blood vessel image and include multiple consecutive pixels (or voxels) in the blood vessel image. In some embodiments, each of the image patches may be centered on a center point of blood vessels in the blood vessel image. The center point refers to a point at a center of one blood vessel. For example, the center point may be a point at a centerline of the blood vessel.

In some embodiments, the processing device 140 may obtain a segmentation result by performing a blood vessel segmentation on the blood vessel image. The segmentation result may indicate the blood vessels in the blood vessel image. For example, the processing device 140 may perform the blood vessel segmentation on the blood vessel image using an image segmentation algorithm. Exemplary image segmentation algorithms may include a machine learning model-based algorithm (e.g., a U-Net), a threshold segmentation algorithm, a regional growth-based segmentation algorithm, or the like, or any combination thereof.

In some embodiments, the processing device 140 may determine center points of the blood vessels based on the segmentation result. For example, for each of the blood vessels, the processing device 140 may determine the center point of the blood vessel by processing the segmentation result through an image analysis algorithm. Exemplary image analysis algorithms may include a geometric contour-based algorithm (e.g., a bounding box algorithm, an image moment algorithm, etc.), a skeleton-based algorithm, a distance transformation algorithm, a connected component analysis algorithm, or the like, or any combination thereof. As another example, for each of the blood vessels, the processing device 140 may determine a centerline of the blood vessel by performing a centerline extraction operation on the segmentation result, and determine the center point of the blood vessel based on the centerline of the blood vessel. Exemplary centerline extraction operations may include a distance transformation operation, a morphological corrosion operation, an optimum route algorithm, a model-based centerline extraction algorithm, or the like, or any combination thereof.

In some embodiments, the processing device 140 may extract the image patches from the blood vessel image based on the center points of the blood vessels. For example, the processing device 140 may determine a patch size of the image patches, and extract the image patches from the blood vessel image, wherein each image patch has the determined patch size and is centered on one center point of the blood vessels. The patch size may be a length of a side of an image patch, such as a count of pixels (or voxels).

In some embodiments, the patch size of each image patch may be determined based on an actual requirement (e.g., a processing time requirement, a processing memory requirement, a processing accuracy requirement, etc.). More descriptions regarding the determination of the patch size may be found elsewhere in the present disclosure. See, e.g., operation 3254 and relevant descriptions thereof.

In some embodiments, the processing device 140 may determine target center points from the center points of the blood vessels, and extract the image patches from the blood vessel image based on the target center points. For example, the processing device 140 may select some (e.g., 2, 3, 5, etc.) center points from the center points of the blood vessels as the target center points based on importance of the blood vessels, and extract the image patches from the blood vessel image based on the target center points and the patch size.

In 1106, for each of the image patches, the processing device 140 (e.g., the generation module 220) may generate a corrected image patch by inputting the image patch into a trained conditional diffusion model (also referred to as a second conditional diffusion model).

The corrected image patch may be an image patch obtained after the image patch is corrected by the second conditional diffusion model. In some embodiments, the corrected image patch may have an improved quality than the image patch. For example, the corrected image patch may include less motion artifacts than the image patch.

In some embodiments, the second conditional diffusion model may be a trained machine learning model, which can generate the corrected image patch by processing (e.g., correcting) the image patch. For example, the second conditional diffusion model may be the same as or similar to the trained conditional diffusion model as described in FIGS. 3 and 5-8, and the corrected image patch may be the same as or similar to the processed image patch as described in FIGS. 3 and 5-8.

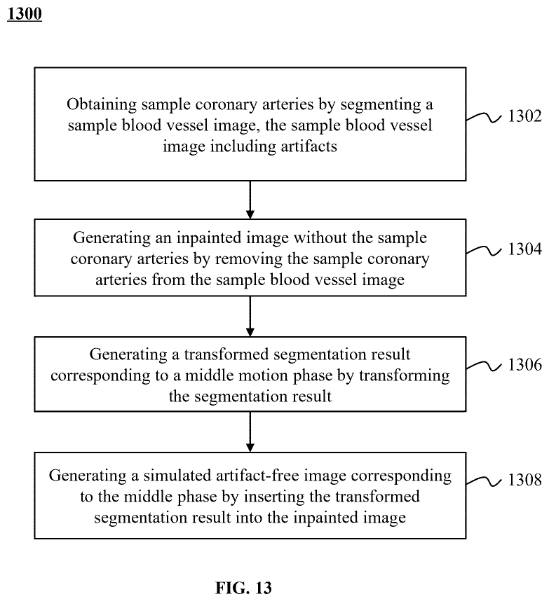

Merely by way of example, the second conditional diffusion model may be obtained by training an initial conditional diffusion model using training samples. Each of the training samples may include a first sample image patch as an input of the initial conditional diffusion model and a corresponding second sample image patch as a label of the initial conditional diffusion model. For example, the processing device 140 may obtain a simulated artifact-corrupted image of sample blood vessels corresponding to a middle motion phase among multiple motion phases and a simulated artifact-free image of the sample blood vessels corresponding to the middle motion phase. The first sample image patch may be extracted from the simulated artifact-corrupted image of sample blood vessels corresponding to the middle motion phase, and the second sample image patch may be extracted from the simulated artifact-free image of the sample blood vessels corresponding to the middle motion phase. The first sample image patch and/or the second sample image patch may be extracted in a similar manner as how the image patch is extracted. More descriptions regarding the generation of the simulated artifact-corrupted image and the simulated artifact-free image may be found elsewhere in the present disclosure. See, e.g., FIGS. 13-16 and relevant descriptions thereof.

As still another example, when the blood vessel image is a 2D image corresponding to an anatomical plane, the input of the second conditional diffusion model may further include one or more adjacent image patches corresponding to the image patch from one or more adjacent blood vessel images of the blood vessel image. For instance, for each of the image patches, the processing device 140 may determine the one or more adjacent image patches corresponding to the image patch from the one or more adjacent blood vessel images of the blood vessel image, and generate the corrected image patch by inputting the image patch and the one or more adjacent image patches into the second conditional diffusion model. The one or more adjacent blood vessel images may correspond to one or more neighboring anatomical planes of the anatomical plane. As another example, the processing device 140 may generate the corrected image patch by inputting the image patch, the one or more adjacent image patches, and a random noise patch into the second conditional diffusion model. More descriptions regarding the one or more adjacent image patches and the generation of the corrected image patch based on the image patch and the one or more adjacent image patches may be found elsewhere in the present disclosure. See, e.g., FIG. 6 and relevant descriptions thereof.

As yet another example, the processing device 140 may generate 180° parallel-beam sinogram data by performing forward projection on the image patch, divide the 180° parallel-beam sinogram data into multiple sinogram datasets corresponding to different partial angular ranges, and generate partial angle reconstruction (PAR) image blocks by performing back-projection on the sinogram datasets. Then, the PAR image blocks may be input into the second conditional diffusion model to generate the corrected image patch. Correspondingly, each of the training samples may include sample PAR image blocks corresponding to the first sample image patch. More descriptions regarding the generation of the corrected image patch based on the PAR image blocks may be found elsewhere in the present disclosure. See, e.g., FIG. 9 and relevant descriptions thereof.

As yet another example, the processing device 140 may obtain a deformation vector field (DVF) by inputting the image patch into the second conditional diffusion model, and generate the corrected image patch by processing the DVF and the image patch using a spatial transformer layer (STL). At this time, the second conditional diffusion model may be trained end-to-end within a supervised learning framework that incorporates the initial conditional diffusion model and the STL. More descriptions regarding the generation of the corrected image patch based on the DVF and the image patch may be found elsewhere in the present disclosure. See, e.g., FIG. 10 and relevant descriptions thereof.

In 1108, the processing device 140 (e.g., the generation module 220) may generate a corrected blood vessel image based on the blood vessel image and the corrected image patches.

In some embodiments, the corrected blood vessel image may have an improved quality than the blood vessel image. For example, the corrected blood vessel image may include less motion artifacts than the blood vessel image. For instance, the corrected blood vessel image may be an artifact-free image, and the blood vessel image may be an artifact-corrupted image.

In some embodiments, the processing device 140 may generate the corrected blood vessel image by correcting the blood vessel image based on the corrected image patches. For example, the processing device 140 may replace corresponding image patches in the blood vessel image with the corrected image patches to generate the corrected blood vessel image.

For illustration purposes, a CCTA image is taken as an example and described below.

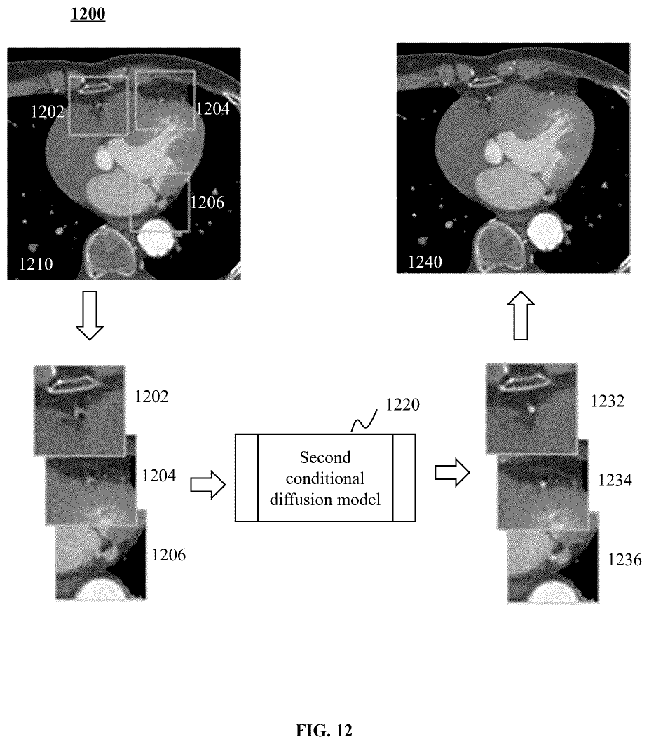

Referring to FIG. 12, FIG. 12 is a schematic diagram illustrating an exemplary process 1200 for generating a corrected CCTA image according to some embodiments of the present disclosure.

As illustrated in FIG. 12, image patches 1202, 1204, and 1206 are extracted from a CCTA image 1210. Each of the image patches 1202, 1204, and 1206 are centered on a center point of blood vessels in the CCTA image 1210. The image patch 1202 is input into a second conditional diffusion model 1220, and the second conditional diffusion model 1220 outputs a corrected image patch 1232. The image patch 1204 is input into the second conditional diffusion model 1220, and the second conditional diffusion model 1220 outputs a corrected image patch 1234. The image patch 1206 is input into the second conditional diffusion model 1220, and the second conditional diffusion model 1220 outputs a corrected image patch 1236. Further, a corrected CCTA image 1240 is generated by replacing the image patches 1202, 1204, and 1206 in the CCTA image 1210 with the corrected image patches 1232, 1234, and 1236, respectively. The corrected CCTA image 1240 has an improved quality than the CCTA image 1210.

According to some embodiments of the present disclosure, the second conditional diffusion model can be employed to correct the blood vessel image (e.g., the CCTA image), thereby reducing or eliminating the motion artifacts in the blood vessel image, and improving the quality of the blood vessel image. Furthermore, the image patches centered on the center points of the blood vessels in the blood vessel image can be extracted and subsequently corrected using the trained conditional diffusion model to generate the corresponding corrected image patches. The image patches instead of the entire blood vessel image are input into the trained conditional diffusion model, therefore, the computational resources required for the application of the trained conditional diffusion model can be significantly reduced, thereby enhancing computational efficiency. In addition, the trained conditional diffusion model can process the image patches without requiring the sinogram data, the visualization of the blood vessels can be improved, thereby enhancing the accuracy of the diagnostic assessment.

FIG. 13 is a flowchart illustrating an exemplary process 1300 for generating a simulated artifact-free image according to some embodiments of the present disclosure.

In 1302, the processing device 140 (e.g., the generation module 220) may generate a segmentation result of sample blood vessels by segmenting a sample blood vessel image of the sample blood vessels.

The sample blood vessel image refers to a blood vessel image for generating a training sample used to train a second conditional diffusion model. In some embodiments, the sample blood vessel image may include artifacts (e.g., motion artifacts caused by the physiological motion).

The sample blood vessel image may be obtained in a similar manner as how the blood vessel image is obtained as described in operation 902. For example, the processing device 140 may obtain the sample blood vessel image from an imaging device (e.g., the imaging device 110) or a storage device (e.g., the storage device 150, a database, or an external storage) that stores the sample blood vessel image.

The segmentation result (also referred to as a sample segmentation result) of the sample blood vessels may be generated by performing a blood vessel segmentation on the blood vessel image. More descriptions regarding the blood vessel segmentation may be found elsewhere in the present disclosure. See, e.g., FIG. 11 and relevant descriptions thereof.

In 1304, the processing device 140 (e.g., the generation module 220) may generate an inpainted image without the sample blood vessels by removing the sample blood vessels from the sample blood vessel image.

In some embodiments, the processing device 140 may remove the sample blood vessels from the sample blood vessel image through an inpainting technique. For example, a region corresponding to the sample blood vessels in the sample blood vessel image is determined based on the segmentation result of the sample blood vessels, and the region is replaced using texture of surrounding tissues of the sample blood vessels through the inpainting technique.

By using the inpainting technique, motions of the sample blood vessels (e.g., cardiac motions of coronary arteries) that exist in the sample blood vessel image can be eliminated, thereby avoiding interference from the motions of the sample blood vessels.

In 1306, the processing device 140 (e.g., the generation module 220) may generate a transformed segmentation result corresponding to a middle motion phase by transforming the segmentation result.

A motion phase refers to a phase relating to a physiological motion of a subject. Exemplary physiological motions may include the cardiac motion, the respiratory motion, random jitter, or the like, or any combination thereof. The cardiac motion refers to a periodic motion of the heart of the subject. The respiratory motion refers to a periodic motion caused by the respiratory of the subject. The random jitter refers to a minor motion of the subject caused by physiological, psychological or habitual factors.

The middle motion phase (also referred to as a middle phase or a static phase) refers to a time point or a brief period in which the physiological motion of the subject (e.g., the myocardium) is relatively static and has a smallest motion amplitude. For example, for the cardiac motion, the middle motion phase is a relatively stable period that occurs after the rapid filling phase and before the end of diastole.

In some embodiments, the processing device 140 may divide the physiological motion into a plurality of motion phases, and generate a deformation vector field (DVF) corresponding to each of the plurality of motion phases. The plurality of motion phases may correspond to different locations of the sample blood vessels. A DVF corresponding to a motion phase may include a displacement vector corresponding to each sample vascular point on the sample blood vessels.

In some embodiments, a dimension of the DVF may relate to a dimension of the sample blood vessel image. For example, if the sample blood vessel image is a 2D image, the DVF may be a 2D DVF. As another example, if the sample blood vessel image is a 3D image, the DVF may be a 3D DVF.

In some embodiments, a count of the plurality of motion phases (or the DVFs) may be determined based on a system default setting or set manually by a user. For example, the count of the plurality of motion phases may be 10, 20, 30, 40, 60, etc. Alternatively, the count of the plurality of motion phases may be determined based on an accuracy requirement and/or an efficiency requirement. For example, the larger the count of the motion phases is, the closer to the true sampling may be, the higher the accuracy may be, but the lower the efficiency may be.

By transforming the segmentation result based on the DVF corresponding to each of the plurality of motion phases, the physiological motion of the segmentation result can be simulated.

For example, the processing device 140 may simulate the cardiac motion during systole and diastole through an elastic deformation field (referring to phase parameters of the cardiac motion cycle). As another example, the processing device 140 may superimpose a Gaussian deformation field to simulate a diaphragm displacement (e.g., a motion amplitude is within a range from 3 mm to 8 mm, and a frequency is within a range from 0.2 Hz to 0.5 Hz). As still another example, the processing device 140 may simulate the random jitter by adding a high-frequency and low-amplitude jitter (e.g., a motion amplitude is within a range from 1 mm to 3 mm, and a frequency is larger than 1 Hz).

In some embodiments, the processing device 140 may determine a DVF corresponding to the middle motion phase, and transform the segmentation result based on the DVF corresponding to the middle motion phase to generate the transformed segmentation result.

For example, the DVF corresponding to the middle motion phase may include a first DVF, a second DVF, a third DVF, or the like, or any combination thereof. The first DVF may be related to the cardiac motion in the middle motion phase, the second DVF may be related to the respiratory motion in the middle motion phase, and the third DVF may be related to random jitter.

For instance, the processing device 140 may determine the first DVF by determining a first motion direction and a first displacement of each sample vascular point based on the amplitude and direction of the cardiac motion in the middle motion phase, determine the second DVF by determining a second motion direction and a second displacement of each sample vascular point based on the amplitude and direction of the respiratory motion in the middle motion phase, and determine the third DVF by randomly generating the random jitter.

In some embodiments, the processing device 140 may determine a weight for each of the first DVF, the second DVF, and the third DVF. For example, for each sample vascular point, the processing device 140 may determine the weight of the first DVF and the weight of the second DVF based on a positional relationship between the sample vascular point and the heart, and a positional relationship between the sample vascular point and the lungs. As another example, the processing device 140 may determine the weight of the third DVF based on information (e.g., medical history) of the subject.

As another example, for each of the motion phases, the processing device 140 may determine a DVF corresponding to the motion phase, and generate a transformed segmentation result corresponding to the motion phase by transforming the segmentation result based on the DVF corresponding to the motion phase. Further, the processing device 140 may generate the transformed segmentation result corresponding to the middle motion phase by averaging the transformed segmentation results corresponding to the motion phases.

In 1308, the processing device 140 (e.g., the generation module 220) may generate a simulated artifact-free image corresponding to the middle motion phase by inserting the transformed segmentation result into the inpainted image.

The simulated artifact-free image refers to a simulated image of the sample blood vessels without artifacts or with limited artifact. Since the transformed segmentation result corresponds to the middle motion phase in which the physiological motion of the subject is relatively static and has a smallest motion amplitude, it can be regarded as an artifact-free segmentation result, and thus the image generated by inserting the transformed segmentation result into the inpainted image can be regarded as the simulated artifact-free image.

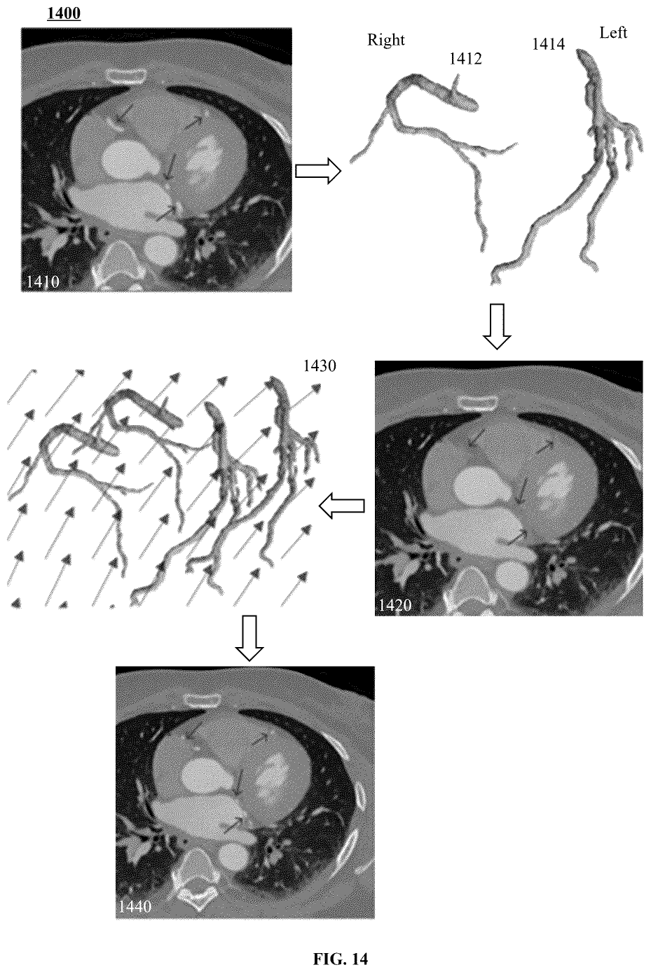

Referring to FIG. 14, FIG. 14 is a schematic diagram illustrating an exemplary process 1400 for generating a simulated artifact-free image according to some embodiments of the present disclosure.

As illustrated in FIG. 14, a segmentation result of the left and right coronary arteries 1412 and 1414 is generated by segmenting a sample blood vessel image 1410 of the left and right coronary arteries 1412 and 1414. The sample blood vessel image 1410 includes artifacts. An inpainted image 1420 without the left and right coronary arteries 1412 and 1414 is generated by removing the left and right coronary arteries 1412 and 1414 from the sample blood vessel image 1410. A transformed segmentation result 1430 corresponding to a middle motion phase may be generated by transforming the segmentation result of the left and right coronary arteries 1412 and 1414. Finally, a simulated artifact-free image 1440 corresponding to the middle phase is generated by inserting the transformed segmentation result 1430 into the inpainted image 1420.

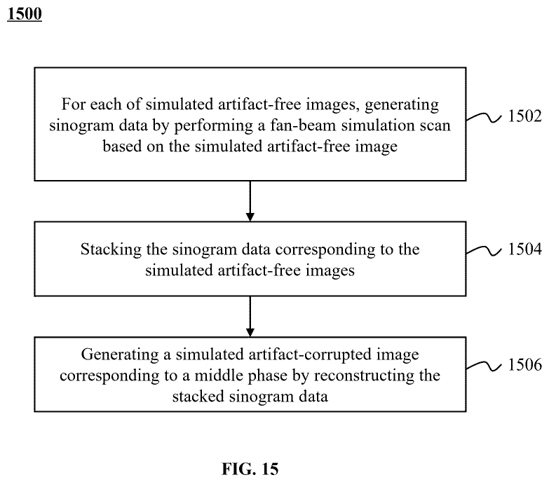

FIG. 15 is a flowchart illustrating an exemplary process 1500 for generating a simulated artifact-corrupted image according to some embodiments of the present disclosure.

In 1502, for each of simulated artifact-free images, the processing device 140 (e.g., the generation module 220) may generate sinogram data by performing a fan-beam simulation scan based on the simulated artifact-free image.

In some embodiments, the processing device 140 may obtain the simulated artifact-free images corresponding to a plurality of motion phases. The simulated artifact-free images may be obtained in a similar manner as how the simulated artifact-free image corresponding to the middle motion phase is generated as described in FIGS. 13 and 14.

The fan-beam simulation scan refers to a mathematical operation simulating a physical process of CT scanning using fan-beams. For example, for each simulated artifact-free image corresponding to one motion phase, the processing device 140 may generate corresponding sinogram data (projection data) covering an angular range of a degrees by performing the fan-beam simulation scan based on the simulated artifact-free image. For instance, if a total scanning angle of the fan-beam simulation scan is 2400 (or an equivalent 240° range), and a count of the plurality of motion phases is 40, each motion phases may correspond to 6° (i.e., a degrees is 6°).

In 1504, the processing device 140 (e.g., the generation module 220) may stack the sinogram data corresponding to the simulated artifact-free images.

For example, the processing device 140 may stack the sinogram data corresponding to the plurality of motion phases in chronological order to form stacked sinogram data. Since sample blood vessels have different positions corresponding to the plurality of motion phases, the stacked sinogram data may contain projection inconsistencies, thereby simulating real motion artifacts. Therefore, the stacked sinogram data may also be referred to as short-time scanning sinogram data with motion contamination/motion artifacts.

In 1506, the processing device 140 (e.g., the generation module 220) may generate a simulated artifact-corrupted image corresponding to a middle motion phase by reconstructing the stacked sinogram data.

For example, the processing device 140 may reconstruct the stacked sinogram data using an image reconstruction algorithm to generate the simulated artifact-corrupted image corresponding to the middle motion phase. As described in connection with 1504, the staked sinogram data is generated by stacking the sinogram data corresponding to the plurality of motion phases in chronological order, and it can be regarded as corresponding to an average of the sinogram data and the middle motion phase. Thus, the reconstruction result of the staked sinogram data (i.e., the simulated artifact-corrupted image) can also be regarded as corresponding to the middle motion phase. Exemplary image reconstruction algorithms may include an analytical algorithm (e.g., a filtered back-projection (FBP) algorithm), an iteration algorithm, a maximum likelihood expectation-maximization (MLEM) algorithm, an ordered subset expectation maximization (OSEM) algorithm, a least squares (LS) algorithm, or the like, or any combination thereof. For example, the stacked sinogram data may be reconstructed using the FBP algorithm with Parker weights.

In some embodiments, the processing device 140 may perform the motion simulation based on real motion data. For instance, real motion artifact data may be collected, e.g., through low-dose CT dynamic sequences, and fused with the stacked sinogram data. As another example, a motion artifact transfer network (e.g., a CycleGAN structure) may be developed to transfer the stacked sinogram data (e.g., simulated artifact features) to sinogram data with real image data. Further, the processing device 140 may reconstruct the sinogram data with real image data using the image reconstruction algorithm to generate the simulated artifact-corrupted image corresponding to the middle motion phase.

In some embodiments, the processing device 140 may determine the simulated artifact-free image corresponding to the middle motion phase and the simulated artifact-corrupted image corresponding to the middle motion phase of the sample blood vessels as a training sample for training an initial conditional diffusion model. Alternatively, the processing device 140 may determine the training sample based on the simulated artifact-free image corresponding to the middle motion phase and the simulated artifact-corrupted image corresponding to the middle motion phase. For example, the processing device 140 may extract a first sample image patch from the simulated artifact-corrupted image corresponding to the middle motion phase, and a second sample image patch from the simulated artifact-free image corresponding to the middle motion phase. Further, the processing device 140 may determine the first sample image patch and the second sample image patch as the training sample. As another example, the processing device 140 may generate a first shifted sample image by performing, based on a randomly generated sample shift vector, a circular shift on the simulated artifact-corrupted image, and generate a second shifted sample image by performing, based on the sample shift vector, a circular shift on the simulated artifact-free image. Further, the processing device 140 may extract the first sample image patch from the first shifted sample image and the corresponding second sample image patch from the second shifted sample image, and determine the first sample image patch and the second sample image patch as the training sample.

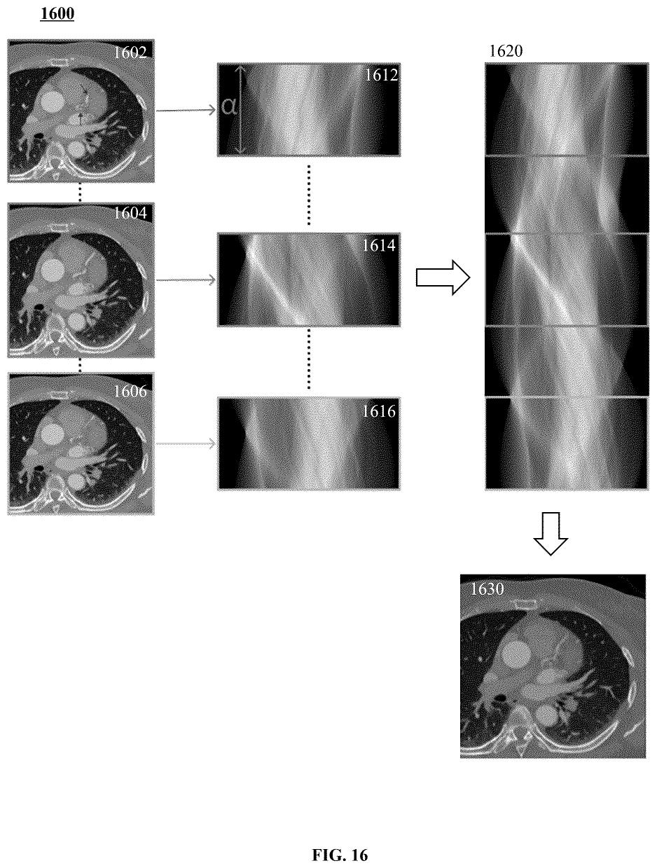

Referring to FIG. 16, FIG. 16 is a schematic diagram illustrating an exemplary process 1600 for generating a simulated artifact-corrupted image according to some embodiments of the present disclosure.

As illustrated in FIG. 16, simulated artifact-free images may include a simulated artifact-free image 1602 corresponding to a first motion phase, a simulated artifact-free image 1604 corresponding to a second motion phase, a simulated artifact-free image 1606 corresponding to a third motion phase. Sinogram data 1612 covering an angular range of a degrees may be generated by performing a fan-beam simulation scan based on the simulated artifact-free image 1602, sinogram data 1614 may be generated by performing a fan-beam simulation scan based on the simulated artifact-free image 1604, and sinogram data 1616 may be generated by performing a fan-beam simulation scan based on the simulated artifact-free image 1606. Stacked sinogram data 1620 may be generated by stacking the sinogram data 1612, 1614, 1616, etc. Further, a simulated artifact-corrupted image 1630 corresponding to a middle motion phase may be generated by reconstructing the stacked sinogram data 1620.

According to some embodiments, training inputs (e.g., the first sample image patch) and labels (e.g., the second sample image patch) for training the initial conditional diffusion model can be generated through simulation, thereby ensuring the accuracy of the training samples and improving the training accuracy of the conditional diffusion model.

Merely by way of example, a trained conditional diffusion model was obtained as below.

Dataset: 16 clinical cardiac datasets were acquired on a United Imaging uCT Atlas system with a circular cone-beam geometry. Data were acquired with an approved IRB and de-identified for later use. The DICOM images obtained from vendor's motion-compensation algorithm, named CardioCapture, were used and were re-imaged to create training datasets as described above. Imaging parameters were kept as close as the clinical scans. The source to detector distance is 1200 mm, and the source to ISO center distance is 600 mm. The detector has 556 pixels with 1 mm per pixel. Image reconstruction size is 512×512, and image reconstruction resolution varies from 0.32 mm to 0.43 mm. Out of 16 datasets, 10 datasets (2848 images containing coronary arteries) were used for training, 5 datasets were used for testing, and the last one was hold out for preliminary investigation about the trained diffusion model's performance on clinical cardiac images.

Implementation: The trained conditional diffusion model was implemented in PyTorch and using the same improved U-Net with attention heads. The input channel dimension was 128, and the total number of network parameters was 135 million. A fixed learning rate of 1e-4 was used to train the networks using Adam optimizer for 500 epochs on 128×128 artery patches. Patches were extracted on the fly based on artery masks segmented by a deep learning based segmentation algorithm (e.g., TotalSegmentator) and further randomly shifted with a maximum offset of 8 in both x and y directions. During inference, patches were always centered on the centroids of arteries. Whenever there were overlaps among patches of an image slice, an average was performed before the insertion back to the image. The number of time steps for inference was 128, and the inference time per dataset (650 slices typically) was under 20 minutes on an NVIDIA GeForce RTX 4090 graphics card. The training took about 32 hours with a batch size of 16, which amounts to roughly 20 GB GPU memory.

Experiment: A 2D and a 2.5D diffusion models were individually trained and were compared to a partial image reconstruction (PAR)-based method. This method needs access to sinogram data, where a 240° fan-beam short scan was partitioned into 7 consecutive 60° partial angles with 30° angular overlap. These partial angles were re-binned to parallel-beam data and subsequently reconstructed using FBP with raised cosine weights to yield 7 PAR image blocks. Motion vector fields were estimated between the two conjugate PAR image blocks at 0° and 180°, respectively, and were linearly applied to all 7 PAR image blocks. The resulting PAR image blocks were summed up to obtain the final motion-compensated reconstruction.

To quantify the performance of all comparing methods, their motion-corrected reconstructions along with the motion-corrupted images and ground-truth images were individually segmented using a deep learning based segmentation algorithm (e.g., TotalSegmentator). The two largest connected components in a segmentation were identified as the left and right coronary arteries. Dice score with respect to the ground-truth coronary artery segmentations were computed.

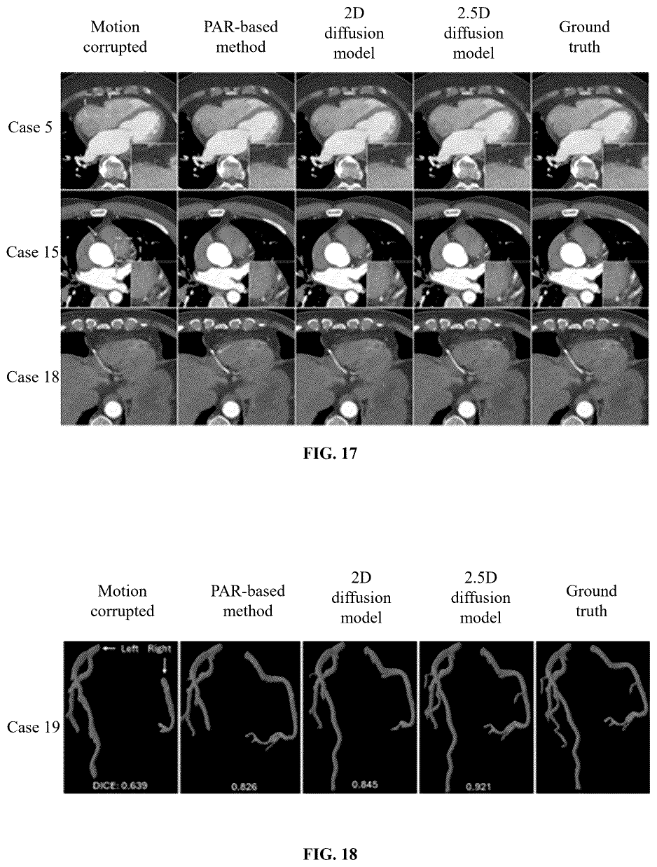

Referring to FIG. 17, FIG. 17 is a schematic diagram illustrating exemplary motion correction results according to some embodiments of the present disclosure.

Motion correction results from three test cases (Case 5, Case 15, and Case 18) are illustrated in FIG. 17. Compared to the PAR-based method, the trained conditional diffusion models are more robust in retaining small arteries (Case 5), reducing complicated motion artifacts (Case 15), and enhancing blurred arteries (Case 18). The 2.5D diffusion model recovers the structures of arteries the best.

For instance, case 5 contains relatively small coronary arteries. The PAR-based method, relying on motion vector fields, sometimes fails to capture small motion-corrupted coronary arteries. In contrast, two diffusion models restore this small artery, and the 2.5D model yields an artery structure most similar to that of the ground truth. In Case 15, the arrows point out two arteries affected (unenhanced or deformed) by mild motions. All approaches were able to restore the underlying round-shaped arteries. However, in the zoom-in region, three nearby arteries show complicated motion artifacts, including shading, distortion, and blurriness. The PAR-based method detects this motion corruption yet still results in distorted arteries. The 2D diffusion model yields two out of three arteries in good shape, and the 2.5D model faithfully recovers all three arteries compared to the ground truth. Lastly, an image slice of Case 18 shows an elongated cross section of an artery being blurred due to motion. The PAR-based method enhances the artery but narrows a segment as pointed by the arrow, leading to a disconnected artery. Both diffusion models enhance the whole artery without narrowness or disconnections.

FIG. 18 is a schematic diagram illustrating exemplary 3D segmentation results according to some embodiments of the present disclosure.

A 3D segmentation of the left and right coronary arteries of Case 19 are illustrated in FIG. 18. All segmentations were obtained using the same segmentation algorithm. Dice scores of the entire 3D segmentation were computed and reported at the bottom. The proposed 2.5D diffusion model yields the most similar artery segmentation to the ground truth and the best Dice score. As can be seen, the segmentation when affected by motions lacks a large portion of the right artery, loses many marginal branches of the left artery, and shows incorrect artery branch diameters. The PAR-based method recovers most of the right artery but fails to retain a long segment of the left artery. Both diffusion models bring back major branches of two coronary arteries, where the 2.5D model performs better in restoring marginal branches. These observations are also aligned with the 3D Dice scores shown at the bottom of each segmentation.

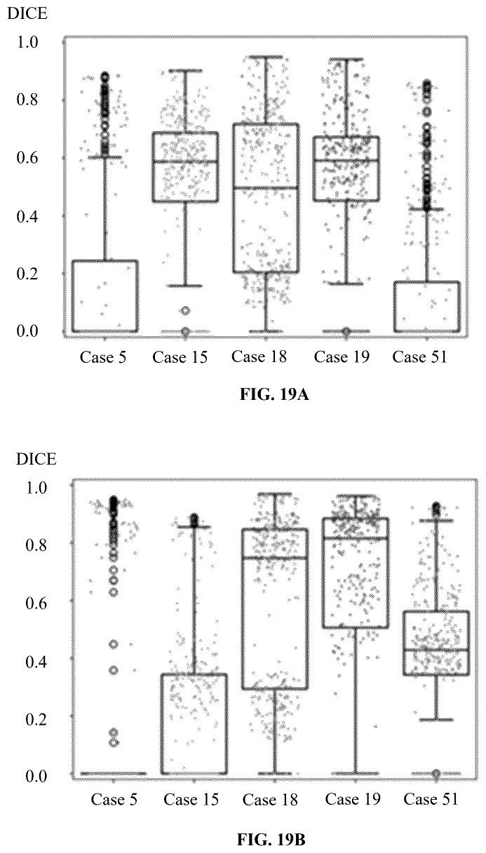

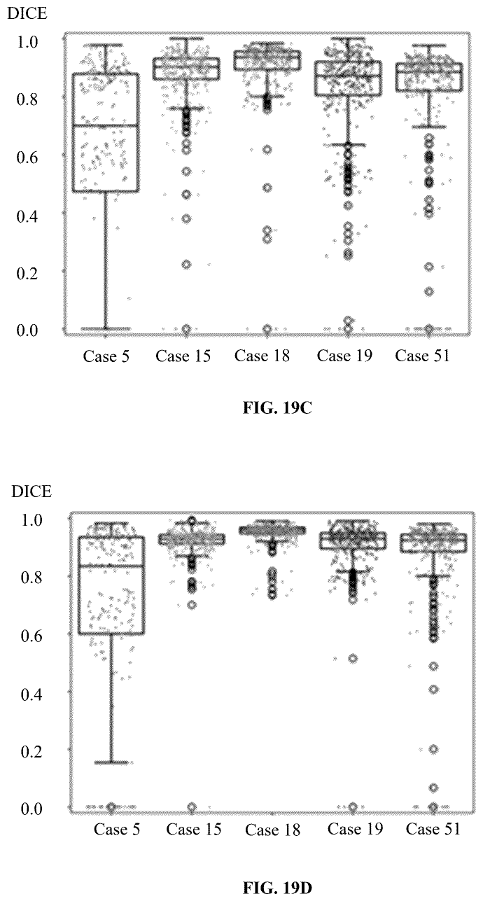

FIGS. 19A-19D are schematic diagrams illustrating box plots with scattered data points of Dice score according to some embodiments of the present disclosure.

Box plots with scattered data points of Dice scores for five test cases of four comparing methods are illustrated in FIGS. 19A-19D. The boxplots of the dice score in FIG. 19A correspond to a motion corrupted method, the boxplots of the dice score in FIG. 19B correspond to a PAR-based method, the boxplots of the dice score in FIG. 19C correspond to a 2D diffusion model, and the boxplots of the dice score in FIG. 19D correspond to a 2.5D diffusion model.

The metric was evaluated on five test cases. The top and bottom of the box represent the 25th and 75th percentiles, and the center line represents the median. Each box was overlaid with data points, representing Dice coefficients of all image slices. Each case has 200 to 300 slices containing simulated coronary arteries. Each data point in the plot represents the Dice score of a slice. The PAR-based method fails in two (Case 5 and Case 15) out of five cases, yielding a lower Dice score than that of motion corrupted images. The 2D diffusion model significantly improves the Dice score, and the 2.5D diffusion model further tightens the spread of Dice score among slices for all five cases. For motion corrupted, the PAR-based method, the 2D diffusion model, and the 2.5D diffusion model, the median (interquartile range) of combined Dice score using all data points are 0.453 (0.653), 0.397 (0.793), 0.890 (0.115), and 0.932 (0.057), respectively.

The training data were created using simulated three-dimensional motions. The training and inference were performed only on coronary artery patches. Test results on five simulated clinical datasets showed the efficacy of the proposed method, outperforming a motion compensation-based method in terms of Dice score of subsequent 3D segmentations of coronary arteries. The incorporation of multiple adjacent slices further improves the efficacy and robustness of motion correction.

It should be noted that the above descriptions regarding the coronal arteries are provided for the purposes of illustration, and are not intended to limit the scope of the present disclosure. For example, the proposed approach may be suitable for other motion corrections if the patch extraction process segments other anatomies, e.g., myocardium.

It should be noted that the descriptions of the processes 300, 500, 600, 900-1100, 1300, and 1500 are provided for the purposes of illustration, and are not intended to limit the scope of the present disclosure. For persons having ordinary skills in the art, various variations and modifications may be conducted under the teaching of the present disclosure. For example, the processes 300, 500, 600, 900-1100, 1300, and 1500 may be accomplished with one or more additional operations not described, and/or without one or more of the operations discussed. Additionally, the order in which the operations of the processes 300, 500, 600, 900-1100, 1300, and 1500 are not intended to be limiting. However, those variations and modifications may not depart from the protection of the present disclosure.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the invention. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate +20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method, implemented on a computing device having at least one processor and at least one storage device, the method comprising:
performing multiple time steps to process a first medical image to generate a second medical image, wherein a current time step among the time steps includes:
performing, based on a randomly generated shift vector, a circular shift on a first intermediate image corresponding to the current time step to generate a second intermediate image, wherein the first intermediate image is generated based on the first medical image or a third intermediate image corresponding to a previous time step;
dividing the second intermediate image into image patches;
generating a third intermediate image corresponding to the current time step based on the image patches, the shift vector, and a trained conditional diffusion model; and
designating the third intermediate image as the second medical image or proceeding to a next time step.

2. The method of claim 1, wherein the generating a third intermediate image corresponding to the current time step based on the image patches, the shift vector, and a trained conditional diffusion model comprises:
for each of the image patches, generating a processed image patch by inputting the image patch into the trained conditional diffusion model;
generating a fourth intermediate image by splicing the processed image patches; and
generating the third intermediate image corresponding to the current time step by performing, based on the shift vector, a reverse circular shift on the fourth intermediate image.

3. The method of claim 2, wherein the generating a processed image patch by inputting the image patch into the trained conditional diffusion model comprises:
generating the processed image patch by inputting the image patch and at least one of positional information or size information of the image patch into the trained conditional diffusion model.

4. The method of claim 2, wherein the first medical image is a two-dimensional (2D) image corresponding to an anatomical plane, and the generating a processed image patch by inputting the image patch into a trained conditional diffusion model comprises:
determining one or more adjacent image patches corresponding to the image patch from one or more adjacent medical images of the first medical image, wherein the one or more adjacent medical images correspond to one or more neighboring anatomical planes of the anatomical plane; and
generating the processed image patch by inputting the image patch and the one or more adjacent image patches into the trained conditional diffusion model.

5. The method of claim 2, wherein the generating a processed image patch by inputting the image patch into the trained conditional diffusion model comprises:
generating 180° parallel-beam sinogram data by performing forward projection on the image patch;
dividing the 180° parallel-beam sinogram data into multiple sinogram datasets corresponding to different partial angular ranges;
generating partial angle reconstruction (PAR) image blocks by performing back-projection on the sinogram datasets; and
generating the processed image patch by inputting the PAR image blocks into the trained conditional diffusion model.

6. The method of claim 2, wherein the generating a processed image patch by inputting the image patch into the trained conditional diffusion model comprises:
obtaining a deformation vector field (DVF) by inputting the image patch into the trained conditional diffusion model; and
generating the processed image patch by processing the DVF and the image patch using a spatial transformer layer (STL), wherein the trained conditional diffusion model is trained end-to-end within a supervised learning framework that incorporates an initial conditional diffusion model and the STL.

7. The method of claim 1, wherein the first intermediate image is generated by concatenating the first medical image or the third intermediate image corresponding to the previous time step with a random noise image.

8. The method of claim 7, wherein the first intermediate image is generated by concatenating the first medical image or the third intermediate image corresponding to the previous time step further with a positional coordinate array corresponding to the first medical image.

9. The method of claim 1, wherein the trained conditional diffusion model is generated by training an initial conditional diffusion model using training samples, each of the training samples includes a first sample image patch and a corresponding second sample image patch, which are generated by:
obtaining a first sample image and a second sample image of a sample subject;
generating a first shifted sample image by performing, based on a randomly generated sample shift vector, a circular shift on the first sample image;
generating a second shifted sample image by performing, based on the sample shift vector, a circular shift on the second sample image; and
extracting the first sample image patch from the first shifted sample image and the corresponding second sample image patch from the second shifted sample image.

10. The method of claim 9, wherein the first sample image and the second sample image are acquired using X-ray imaging,
a loss function used to train the initial conditional diffusion model includes a first loss term and a second loss term,
the first loss term relates to a difference between a predicted image patch output by the initial conditional diffusion model and the second sample image patch, and
the second loss term relates to a difference between back-projection data of the predicted image patch and back-projection data of the first sample image patch.

11. The method of claim 1, wherein the designating the third intermediate image as the second medical image or proceeding to a next time step comprises:
in response to determining that a preset count of time steps has been completed,
evaluating whether the third intermediate image satisfies a quality condition;
in response to determining that the third intermediate image satisfies the quality condition, designating the third intermediate image as the second medical image; or in response to determining that the third intermediate image does not satisfy the quality condition, post-processing the third intermediate image to generate the second medical image or proceeding to the next time step.

12. A method, implemented on a computing device having at least one processor and at least one storage device, the method comprising:
obtaining a blood vessel image;
extracting image patches from the blood vessel image, each of the image patches being centered on a center point of blood vessels in the blood vessel image;
for each of the image patches, generating a corrected image patch by inputting the image patch into a trained conditional diffusion model; and
generating a corrected blood vessel image based on the blood vessel image and the corrected image patches, the corrected blood vessel image having an improved quality than the blood vessel image.

13. The method of claim 12, wherein the blood vessel image is a two-dimensional (2D) image corresponding to an anatomical plane, and for each of the image patches, the generating a corrected image patch by inputting the image patch into a trained conditional diffusion model comprises:
for each of the image patches,
determining one or more adjacent image patches corresponding to the image patch from one or more adjacent blood vessel images of the blood vessel image, wherein the one or more adjacent blood vessel images correspond to one or more neighboring anatomical planes of the anatomical plane; and
generating the corrected image patch by inputting the image patch and the one or more adjacent image patches into the trained conditional diffusion model.

14. The method of claim 12, wherein the generating a corrected image patch by inputting the image patch into a trained conditional diffusion model comprises:
generating 180° parallel-beam sinogram data by performing forward projection on the image patch;
dividing the 180° parallel-beam sinogram data into multiple sinogram datasets corresponding to different partial angular ranges;
generating partial angle reconstruction (PAR) image blocks by performing back-projection on the sinogram datasets; and
generating the corrected image patch by inputting the PAR image blocks into the trained conditional diffusion model.

15. The method of claim 12, wherein the generating a corrected image patch by inputting the image patch into a trained conditional diffusion model comprises:
obtaining a deformation vector field (DVF) by inputting the image patch into the trained conditional diffusion model; and
generating the corrected image patch by processing the DVF and the image patch using a spatial transformer layer (STL), wherein the trained conditional diffusion model is trained end-to-end within a supervised learning framework that incorporates an initial conditional diffusion model and the STL.

16. The method of claim 12, wherein the corrected blood vessel image includes less motion artifacts than the blood vessel image, the trained conditional diffusion model is generated by training an initial conditional diffusion model using training samples, each of the training samples includes a first sample image patch and a second sample image patch,
the first sample image patch is used as a training input and extracted from a simulated artifact-corrupted image of sample blood vessels corresponding to a middle motion phase among multiple motion phases;
the second sample image patch is used as a training label and extracted from a simulated artifact-free image of the sample blood vessels corresponding to the middle motion phase.

17. The method of claim 16, wherein the simulated artifact-free image corresponding to the middle motion phase is generated by:
generating a segmentation result of the sample blood vessels by segmenting a sample blood vessel image of the sample blood vessels, the sample blood vessel image including artifacts;
generating an inpainted image without the sample blood vessels by removing the sample blood vessels from the sample blood vessel image;
generating a transformed segmentation result corresponding to the middle motion phase by transforming the segmentation result; and
generating the simulated artifact-free image corresponding to the middle motion phase by inserting the transformed segmentation result into the inpainted image.

18. The method of claim 17, wherein the sample blood vessels are coronal arteries, and
the segmentation result is transformed based on a first DVF, a second DVF, and a third DVF, the first DVF being related to the cardiac motion in the middle motion phase, the second DVF being related to the respiratory motion in the middle motion phase, and the third DVF being related to random jitter.

19. The method of claim 16, wherein the simulated artifact-corrupted image is generated based on simulated artifact-free images corresponding to the motion phases by:
for each of the simulated artifact-free images, generating sinogram data by performing a fan-beam simulation scan based on the simulated artifact-free image;
stacking the sinogram data corresponding to the simulated artifact-free images; and
generating the simulated artifact-corrupted image corresponding to the middle motion phase by reconstructing the stacked sinogram data.

20. A system, comprising:
at least one storage device including a set of instructions; and
at least one processor in communication with the at least one storage device, wherein when executing the set of instructions, the at least one processor is directed to cause the system to perform operations including:
performing multiple time steps to process a first medical image to generate a second medical image, wherein a current time step among the time steps includes:
performing, based on a randomly generated shift vector, a circular shift on a first intermediate image corresponding to the current time step to generate a second intermediate image, wherein the first intermediate image is generated based on the first medical image or a third intermediate image corresponding to a previous time step;
dividing the second intermediate image into image patches;
generating a third intermediate image corresponding to the current time step based on the image patches, the shift vector, and a trained conditional diffusion model; and
designating the third intermediate image as the second medical image or proceeding to a next time step.
